(19) United States
(12) Patent Application Publication
(10) Pub. No.: US 2023/0225429 A1
(43) Pub. Date: Jul. 20, 2023

(54) WEARABLE ITEMS

(71) Applicant: Rheon Labs Ltd., London (GB)

(72) Inventors: Daniel James Plant, London (GB); Sophie Rebecca Owen, London (GB); Roman Chaika, London (GB)

(21) Appl. No.: 18/123,167

(22) Filed: Mar. 17, 2023

Related U.S. Application Data
(63) Continuation of application No. PCT/GB21/52424, filed on Sep. 17, 2021.

(30) Foreign Application Priority Data
Sep. 18, 2020 (GB) 2014726.0

(51) Int. Cl. A41D 13/00; A41B 11/00; A41B 17/00; A41C 3/00; A41D 1/089; A41D 13/015; A41D 27/10; A41D 31/18; A63B 69/00

(52) U.S. Cl. CPC A41D 13/0015; A41B 11/003; A41B 17/00; A41C 3/0057; A41D 1/089; A41D 13/0156; A41D 27/10; A41D 31/185; A63B 69/0059

(57) ABSTRACT

Wearable item comprising a motion control system and a method of manufacturing thereof. The wearable item comprises a body-close wearable item which, when worn by a user, at least a part of the wearable item is positioned adjacent to the body of the user. The motion control system comprises at least one layer of strain-rate sensitive material configured to control motion of one or more body parts of the user.

### CROSS-REFERENCE TO RELATED APPLICATIONS

This Application is a continuation of International Patent Application No. PCT/GB2021/052424, filed on Sep. 17, 2021, which claims priority to GB Application No. 2014726.0, filed on Sep. 18, 2020, the entire contents of each of which are hereby incorporated by reference.

### INTRODUCTION

### Technical Field

The present disclosure concerns wearable items. More particularly, but not exclusively, this disclosure concerns a wearable item comprising a motion control system. The disclosure also concerns a method of manufacturing a wearable item comprising a motion control system.

### Background

The present disclosure relates to body-close wearable items for use during exercise. Examples of such wearable items include compression garments, sports bras, and kinesiology tape.

Hamstring strain injuries (HSI) are common in sports involving sprinting and jumping. During high speed running, the biceps femoris long head is the muscle most frequently injured, often where the muscle fibers join the tendon. The severity of the strain can vary from mild to a complete tear of the muscle. HSI often occur as a result of muscle overstretching and/or absorption of energy from the decelerating limb whilst the muscles are lengthening. It is accepted that injury severity can be reduced or prevented entirely by altering an athletes' range of motion (ROM), reducing the demands on muscles, or by reducing soft tissue oscillations and vibration during activity. Therefore, controlling the muscle to reduce the range of motion to just the axial direction may reduce the risk of injury, as the muscle will not swing in the circumferential/radial direction adding strain to the tendon and increasing the risk of tearing/snapping. Compression garments can help to prevent HSI and improve performance by exerting global and or local pressure on the soft tissue. The pressure limits ROM, reduces soft tissue oscillations and accelerates muscle oxygenation.

Compression garments, by virtue of their mode of operation, are also inherently difficult for a user to put on and take off, as they are designed to be narrower than the body part on which they are to be worn, such that the garment applies pressure to the body part when worn.

Kinesiology tapes (KT) and athletic tapes are used in therapy and to enhance sporting performance and are applied over injury-prone or rehabilitating areas of the body following kinesiology principles. Although physiological improvements are seen when kinesiology tape is worn, the exact physiological effect of the tape is not known. It has been found that kinesiology tape can have beneficial effects on oedema, muscular performance and facilitation, proprioception, balance, and pain. Kinesiology tape works in a similar manner to compression garments by limiting and controlling soft-tissue movement to reduce the risk of injury.

The present disclosure seeks to mitigate the above-mentioned problems. Alternatively or additionally, the present disclosure seeks to provide improved body-close wearable items for use during exercise.

### SUMMARY

According to a first aspect of the present disclosure, there is provided a wearable item comprising a motion control system, wherein the wearable item comprises a body-close wearable item which, when worn by a user, at least a part of the wearable item is positioned adjacent to the body of the user, and wherein the motion control system comprises at least one layer of strain-rate sensitive material configured to control motion of one or more body parts of the user.

According to a second aspect of the disclosure there is also provided a method of manufacturing a wearable item comprising a motion control system, the method comprising: forming the wearable item comprising a body-close wearable item which, when worn by a user, at least a part of the wearable item is positioned adjacent to the body of the user; and forming the motion control system comprising at least one layer of strain-rate sensitive material configured to control motion of one or more body parts of the user.

It will of course be appreciated that features described in relation to one aspect of the present disclosure may be incorporated into other aspects of the present disclosure. For example, methods of the disclosure may incorporate any of the features described with reference to apparatus of the disclosure and vice versa.

### DESCRIPTION OF THE DRAWINGS

Embodiments of the present disclosure will now be described by way of example only with reference to the accompanying schematic drawings of which:

FIG. 1 shows a perspective view of a human lower leg according to embodiments of the present disclosure;

FIG. 2 shows a chart of the resultant root mean squared acceleration of a user's muscles when wearing both non-compression shorts and compression shorts of the prior art.

FIG. 3 shows a perspective view of a sports bra on a user according to embodiments of the present disclosure;

FIG. 4 shows configurations of motion control systems for shorts according to embodiments of the present disclosure;

FIGS. 4a and 4b show configurations of motion control systems for tights according to embodiments of the present disclosure;

FIGS. 5a, 5b, 5c, and 5d show configurations of motion control systems for sports bras according to embodiments of the present disclosure;

FIGS. 6a and 6b show configurations of motion control systems for kinesiology tape according to embodiments of the present disclosure;

FIGS. 7a and 7b show perspective views of each of the kinesiology tape configurations of FIGS. 6a and 6b applied to the thigh of a user according to embodiments of the present disclosure;

FIG. 8a shows uniaxial stress-strain tensile deformation curves for a strain rate sensitive material according to embodiments of the present disclosure;

FIG. 8b shows uniaxial tension hysteresis curves for a strain rate sensitive material according to embodiments of the present disclosure;

FIG. 9a shows a chart of the energy absorption of strain rate sensitive substance according to embodiments of the present disclosure and a TPU film laminate material;

FIG. 9b shows a load strain curve of a wearable item laminated with the strain rate sensitive substance according to embodiments of the present disclosure;

FIG. 9c shows a chart of energy absorption of strain rate sensitive substance laminated onto fabric according to embodiments of the present disclosure, TPU laminated onto fabric, and the fabric alone;

FIG. 10 shows a graph of the axial acceleration of a user's hamstring muscle when wearing compressions shorts of the prior art and when wearing compression shorts according to embodiments of the present disclosure;

FIG. 11 shows a graph of the circumferential acceleration of the user's hamstring muscle of FIG. 10;

FIG. 12 shows a graph of the resultant acceleration of the user's hamstring muscle of FIGS. 10 and 11;

FIG. 13 shows a graph of the resultant accelerations of the user's hamstring muscle of FIGS. 10 to 12 during the swing phase;

FIG. 14 shows a chart of the resultant RMS acceleration of a user's hamstring muscle when wearing compression shorts having different geometries of planar motion controlling cells according to embodiments of the present disclosure;

FIG. 15 shows a graph of the resultant RMS acceleration of the hamstring muscles of two users with different levels of muscle mass when wearing compression shorts having different geometries of planar motion controlling cells according to embodiments of the present disclosure;

FIG. 15a shows a chart of the reduction of the resultant RMS acceleration of the quadriceps and hamstring of a user when running at various speeds;

FIG. 16 shows a graph comparing accelerations of a user's hamstring when wearing compression shorts of the prior art to when wearing shorts according to embodiments of the present disclosure;

FIG. 17 shows a graph of the resultant RMS acceleration of a user's hamstring when wearing compression shorts according to embodiments of the present disclosure, along with the total area of strain rate sensitive material in each pair of shorts;

FIG. 18 shows a graph of the perceived support given by shorts according to embodiments of the present disclosure;

FIG. 19 shows a graph of the magnitude with respect to frequency of the accelerations of a user's hamstring when wearing compression shorts of the prior art and shorts according to embodiments of the present disclosure;

FIG. 20 shows a graph of the magnitude with respect to frequency of the accelerations of the user's hamstring of FIG. 19 at main body running frequencies;

FIG. 21 shows a graph of the acceleration magnitude with respect to frequency of the accelerations of the user's hamstring of FIG. 19 at higher muscle “wobble” frequencies;

FIG. 21a shows a chart of the weighted power output of a user wearing motion control tights according to embodiments of the present disclosure;

FIG. 22 shows a chart of the resultant RMS acceleration of a muscle of a user of kinesiology tape according to embodiments of the present disclosure when applied directly onto the skin and when applied onto a compression garment;

FIG. 23 shows a chart of the resultant RMS acceleration of a user's muscle when using kinesiology tape according to embodiments of the present disclosure;

FIG. 24 shows a chart of the accelerations of a user's muscle when using kinesiology tape according to embodiments of the present disclosure;

FIG. 24a shows a chart of the energy absorption of motion control tape according to embodiments of the present disclosure at a number of stretch speeds;

FIGS. 24b shows a chart of the stiffness of motion control tape according to embodiments of the present disclosure at a number of stretch speeds;

FIG. 24c shows hysteresis curves of baseline motion control tape at a number of stretch speeds;

FIG. 24d shows a hysteresis curve of motion control tape according to embodiments of the present disclosure at a number of stretch speeds;

FIG. 24e shows a chart comparing the energy absorption of motion control tapes of FIGS. 24c and 24d;

FIG. 24f shows a chart comparing the stiffness of motion control tapes of FIGS. 24c and 24d;

FIG. 25 shows a graph of the axial and circumferential displacement of the breast tissue of a user when wearing a sports bra of the prior art and sports bras according to embodiments of the present disclosure;

FIG. 26 shows a graph of resultant displacement of the breast tissue of the user of FIG. 25 according to embodiments of the present disclosure;

FIG. 27 shows a chart of the axial RMS displacement of the breast tissue of a user when wearing sports bras according to embodiments of the present disclosure whilst running at 10 km/hr;

FIG. 28 shows a chart of the circumferential RMS displacement of the breast tissue of the user of FIG. 27 according to embodiments of the present disclosure;

FIG. 29 shows a chart of the axial RMS displacement of the breast tissue of a user when wearing sports bras according to embodiments of the present disclosure whilst running at 13 km/hr;

FIG. 30 shows a chart of the circumferential RMS displacement of the breast tissue of the user of FIG. 29 according to embodiments of the present disclosure;

FIG. 31 shows charts of the circumferential and axial RMS displacements of the breast tissue of a user when wearing sports bras according to embodiments of the present disclosure whilst walking at 6 km/hr;

FIG. 32 shows a chart of the axial RMS displacement of the breast tissue of a user when wearing sports bras according to embodiments of the present disclosure whilst star jumping;

FIG. 32a shows a chart of the axial RMS displacement of the breast tissue of a user when wearing sports bras according to embodiments of the present disclosure;

FIG. 32b shows a graph of the energy absorption of motion control tapes according to embodiments of the present disclosure having differing area coverage of SRS material and at a number of stretch speeds;

FIG. 32c shows a graph of the stiffness of motion control tapes according to embodiments of the present disclosure having differing area coverage of SRS material and at a number of stretch speeds;

FIG. 32d shows a chart of the energy absorption of motion control tape according to embodiments of the present disclosure having motion controlling cells in different orientations at a number of stretch speeds;

FIG. 32e shows a chart of the stiffness of motion control tape according to embodiments of the present disclosure having motion controlling cells in different orientations at a number of stretch speeds;

FIG. 33 shows the perceived support given by sports bras according to embodiments of the present disclosure;

FIG. 33a shows a chart of the average perceived score for ease of donning and doffing of sports bras according to embodiments of the present disclosure;

FIG. 33b shows chart of the average perceived comfort of sports bras according to embodiments of the present disclosure;

FIG. 33c shows a chart of the perceived support, comfort, breathability, and power of tights according to embodiments of the present disclosure;

FIG. 34 shows charts of the axial and resultant RMS displacements of the breast tissue of a user when wearing sports bras according to embodiments of the present disclosure, along with the total area of strain rate sensitive material in each bra;

FIG. 34a shows a chart of axial RMS displacement of the breast tissue of a user when wearing sports bras according to embodiments of the present disclosure and the area coverage of strain-rate sensitive material for those sports bras;

FIG. 35 shows the flow chart illustrating the steps of a method according to embodiments of the present disclosure;

FIG. 36 shows a graph of the perceived support given by garments according to embodiments of the present disclosure, along with the total area of strain rate sensitive material in each garment; and

FIG. 37 shows a graph of the perceived support given by sports bras according to embodiments of the present disclosure, along with the total area of strain rate sensitive material in each sports bra.

### DETAILED DESCRIPTION

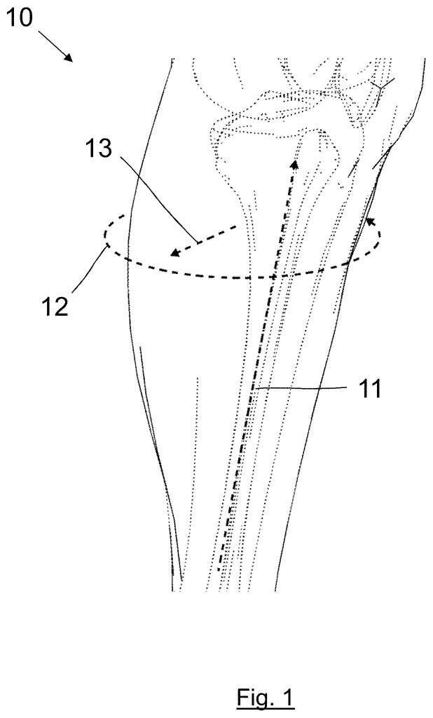

FIG. 1 shows a perspective view of a human lower leg 10. The effectiveness of compression shorts can be evaluated by measuring hamstring soft tissue accelerations in the axial direction 11 (i.e. along the bone), the circumferential direction 12 (i.e. around the bone), and the radial direction 13 (i.e. towards/away from the bone).

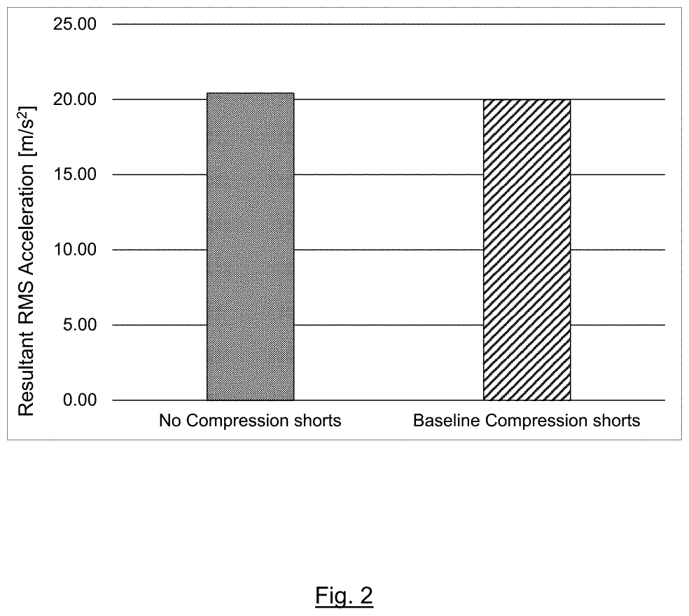

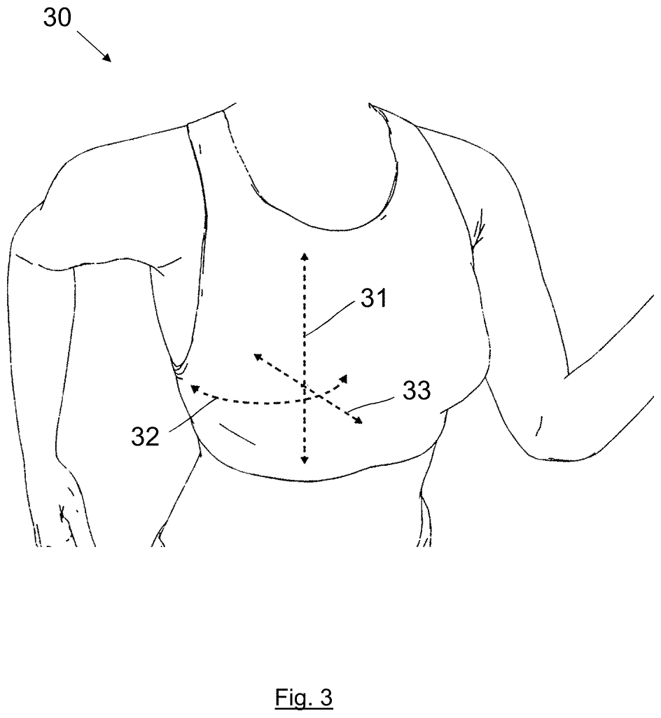

FIG. 2 shows a chart of the resultant root mean squared acceleration of a user's hamstring muscle for both non-compression shorts and compression shorts. When wearing the non-compression shorts, the user experiences muscle acceleration of 20.42 ms⁻². When wearing the compression shorts, the user experiences muscle acceleration of 19.99 ms⁻² (a reduction of approximately 2%). Thus, the compression shorts provide only a marginal reduction in resultant muscle accelerations compared to non-compressive shorts. Compression shorts therefore provide no significant enhancement of athlete performance or reduction in the probability of hamstring injury.

Running without a sports bra has been found to increase vertical displacement of the breast and exercise-induced breast discomfort, particularly in women with larger breasts. Sports bras of the prior art are either encapsulating or compressive. Encapsulating sports bras have cups which fit around each breast; these are comfortable to wear but provide minimal support. Compressive sports bras are much more supportive, but compromise on comfort and ease of donning and doffing. Historically, manufacturers of sports bra have relied on compressing the breast tissue to be closer to the chest, in order to minimise movement during exercise. This can cause discomfort due to tight fitting bras, particularly on larger busted women. The wide variety of size, shape and density of breast tissue makes producing well-fitting sports bras a complex challenge. Elastic textiles have been widely used in the application of sports bras to allow for differences in breast, shape, size, and weight in order to accommodate a wide range of women. The higher the amount of stretch in the textiles, the more forgiving the fit. However, this can lead to lower levels of support and therefore increased breast tissue movement during exercise. Conversely, tight-fitting bras with lower amounts of stretch can be less comfortable and can compromise the female form. Sports bras, by their nature, are also required to be lightweight and breathable in order to be fit for use in physical activity.

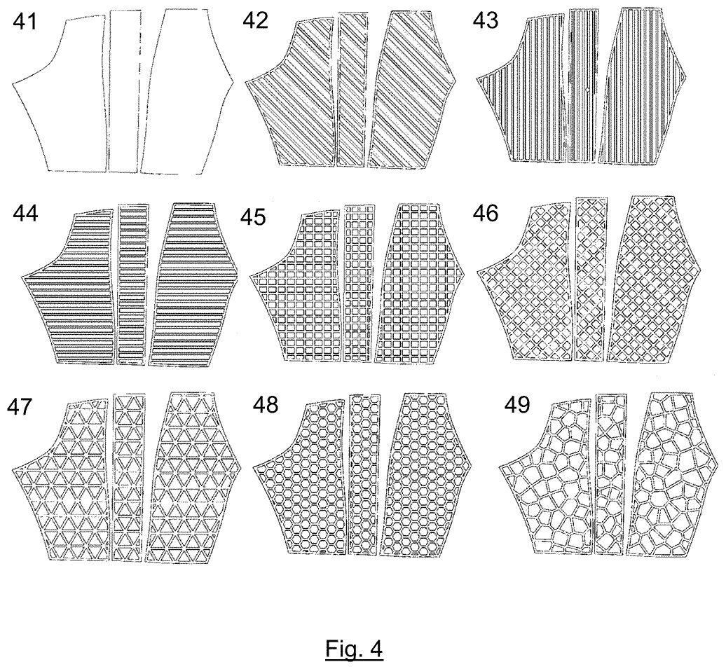

FIG. 3 shows a perspective view of a sports bra 30 on a wearer. The effectiveness of a sports bra can be evaluated by measuring breast tissue accelerations in the axial direction 31 (i.e. along the torso, in the cranial caudal plane), the circumferential direction 32 (i.e. around the torso, in the medial-lateral plane), and the radial direction 33 (i.e. towards/away from the torso, in the anterior-posterior plane).

A first aspect of the present disclosure provides a wearable item comprising a motion control system. It will be understood that, in this context, a motion control system refers to a system that acts to limit and/or damp motion (for example, of a part of the user's body).

The wearable item comprises a body-close wearable item which, when worn by a user, at least a part of the wearable item is positioned adjacent to the body of the user. It will be understood by the skilled person that “body close” refers to a characteristic of the wearable item of, when worn by a user, conforming to the shape of the user's body. Thus, the body-close wearable item can be said to be “skin-tight”. It should be understood that at least some, but not necessarily all, of the wearable item is body-close, i.e. one or more parts or portions of the wearable item are body-close but one or more parts or portions of the wearable item may not be body-close. The skilled person will also appreciate that “adjacent” does not, in this context, require that the wearable item be in direct contact with the wearer's skin. The wearable item may, for example, be worn over another item of clothing. In such cases, the wearable item will nonetheless be adjacent to the user's body by virtue of the wearable item being body-close. The requirement that the wearable item be adjacent to the user's body will therefore be understood by the skilled person to mean that the wearable item, when worn by a user, conforms to the shape of the user's body or part(s) thereof. The skilled person will understand the wearable item to be adjacent to the user's body even where there is a further substance or material positioned between the wearable item and the user.

In embodiments, the wearable item comprises one of: a pair of shorts (such as running shorts), a pair of tights (or leggings), a brassiere (such as a sports bra), a tape, a sock, and a sleeve or tube with an opening at both ends. It will be appreciated that the present disclosure is also applicable to other wearable items.

In embodiments, the wearable item may be configured such that, after having been deformed (for example, by being stretched over a user's body), the wearable item returns to its original shape. Thus, in such embodiments, the wearable item may be body-close by virtue of its elastic properties. In embodiments, the wearable item comprises an elastic material and the wearable item returns to its original shape due to the elasticity of the elastic material.

The motion control system comprises at least one layer of strain-rate sensitive (SRS) material configured to control motion of one or more body parts of the user. A strain rate sensitive material is one which is flexible under low strain rates but, as motion (and therefore strain-rate) increases, becomes less flexible and highly damping, such that it resists the motion. A motion control system incorporating SRS material can therefore be considered to be an “active” motion control system. Thus, in embodiments, the motion control system is configured to control (for example, to limit and/or damp) motion (for example, of the soft-tissue body parts). In embodiments, controlling the motion encompasses controlling velocity and/or displacement and/or accelerations. In such embodiments, it may be that the SRS material is configured to control (for example, to limit and/or damp) the motion. Thus, the wearable item is flexible and easily stretched at low strain rates but is stiffer and more supportive at higher strain rates. This enables easier donning and doffing of the wearable item and also affords the user a normal range of motion (ROM) whilst also affording increased support when the user engages in athletic activity. The stiffness of the SRS material increases in relation to the applied strain-rate, providing more support as the user performs more vigorous physical activity. The damping coefficient of the SRS material also increases with strain rate. Therefore, the SRS material provides motion control by two mechanisms: (i) by providing increased stiffness as strain rate increases, and (ii) by providing increased damping as the strain rate increases.

In embodiments, the at least one layer of strain-rate sensitive material comprises a solid strain-rate sensitive material. The term ‘solid’ here is intended to mean stable in shape and self-supporting (not liquid or fluid).

In embodiments, the at least one layer of strain-rate sensitive material comprises a chemically strain-rate sensitive material. In embodiments, the at least one layer of strain-rate sensitive material comprises a polymer. In embodiments, the at least one layer of strain-rate sensitive material comprises a chemical dilatant.

In embodiments, the one or more body parts comprise soft-tissue body parts. In embodiments, the one or more body parts comprise one or more of: a muscle (for example a hamstring muscle) and a breast. It will be appreciated that a wearable item according to the present disclosure may also be used on other body parts.

In embodiments, controlling motion from movement of one or more body parts of the user comprises controlling velocity of the soft-tissue body parts. In embodiments, the controlling comprises controlling displacement of the soft-tissue body parts. In embodiments, the controlling comprises controlling acceleration of the soft-tissue body parts. In embodiments, the controlling comprises controlling energy absorption (for example, by the wearable item). In embodiments, the controlling comprises controlling stiffness (for example, of the wearable item).

In embodiments, the controlling is dependent on the frequency of motion of the soft-tissue body parts. Thus, the controlling may comprise supressing certain frequencies of movement. In such embodiments, the controlling is greater at relatively high frequencies of motion of the soft-tissue body parts compared to relatively low frequencies of motion of the soft-tissue body parts. Thus, the controlling may comprise supressing relatively high frequencies of movement more than relatively low frequencies. In embodiments, the controlling comprises performing substantially zero control at relatively low frequencies of motion of the soft-tissue body parts. Thus, the controlling may comprise supressing only the relatively high frequencies of movement. In embodiments, the relatively low frequencies comprise frequencies below 5 Hz, or between 1 Hz and 5 Hz. In embodiments, the relatively high frequencies comprise frequencies above 5 Hz, preferably between 10 Hz and 30 Hz. Embodiments in which the controlling is dependent on the frequency of motion can enable the wearable item to constrain undesirable movements of the soft-tissue body part without impeding the desired movement of the soft-tissue body part (for example, the contraction of a muscle) associated with performance of an activity. For example, a runner's muscles will contract with a frequency corresponding to the cadence of the running, but will also undergo higher frequency “wobble.” A wearable item that is configured to suppress specific frequencies of movement may suppress the muscle “wobble” frequencies without impeding the frequencies associated with the muscle contraction.

In embodiments, the at least one layer of strain-rate sensitive material is configured to control motion of the one or more body parts of the user in a given direction. Thus, the wearable item may be configured to suppress movement in one or more specific directions. For example, the strain-rate sensitive material may be configured to allow movement in a first direction (for example, axially along a bone—corresponding to the principal direction of muscle contraction) whilst supressing movement in a second direction (for example, circumferentially around the bone).

In embodiments (for example, where the wearable item comprises a compression garment), the given direction comprises one or both of a radial direction from a bone of the user, and a circumferential direction around a bone. In embodiments where the wearable item comprises a pair of shorts, a tape, a sock, or a sleeve or tube with an opening at both ends, the bone of the user may comprise a femur. It will be appreciated that such wearable items may also be worn on other parts of the body. Such embodiments can constrain movement in the radial and/or circumferential directions, which is associated with an increased risk of injury. Thus, such embodiments may reduce the risk of injury to the user from their activity. In embodiments (for example, where the wearable item comprises a sports bra), the given direction comprises one or more a radial direction from a given body part of the user (for example, the user's torso), an axial direction along the given body part, and a circumferential direction around the given body part. In embodiments where the wearable item comprises a brassiere, the given body part of the user may comprise the torso of the user.

In embodiments, the at least one layer of strain-rate sensitive material is configured not to control motion of the one or more body parts of the user in a different, given direction. In embodiments, the different, given direction comprises an axial direction along a bone of the user. Such embodiments may allow motion in the axial direction, which is associated with muscle contraction, and therefore do not inhibit the physical activity of the user. Meanwhile, movement in the radial and/or circumferential directions, which is associated with an increased risk of injury, is inhibited, reducing the user's risk of injury.

In embodiments, the wearable item comprises a textile layer. In embodiments, the textile layer comprises one or more of knitted textile, woven textile, non-woven textile, and unidirectional fibre textile. In embodiments, it may be that the at least one layer of strain-rate sensitive material is attached to the textile layer. In embodiments, the at least one layer of strain-rate sensitive material is laminated to the textile layer. In embodiments, the at least one layer of strain-rate sensitive material is adhered to the textile layer. In embodiments, the at least one layer of strain-rate sensitive material is woven and/or knitted into the textile layer. In embodiments, the at least one layer of strain-rate sensitive material is heat-pressed onto the textile layer. In embodiments, only a portion of the at least one layer of strain-rate sensitive material is attached to the textile layer. Thus, it may be that at least part of the at least one layer of strain-rate sensitive material is not attached to the textile layer. In such embodiments, it may be that the at least one layer of strain-rate sensitive material is attached to the textile layer at (for example, only at) the edges of the at least one layer of strain-rate sensitive material. In other embodiments, an entire surface of the at least one layer of strain-rate sensitive material is attached to the textile layer. In embodiments, the at least one layer of strain-rate sensitive material is thermoformed to the shape of the garment.

In embodiments, the textile layer may be pre-stretched before the at least one layer of strain-rate sensitive material is attached to it, such that the at least one layer of strain-rate sensitive material is attached to the stretched textile layer. Such embodiments can result in the combined SRS and textile layer varying across its surface in a direction perpendicular to the plane of the textile layer (for example, forcing the textile layer to take on a corrugated form). In embodiments, the SRS and/or the textile layer is embossed or de-bossed to provide such a variation (for example, to have a corrugated form). In embodiments, the combined SRS and textile layer is thermoformed to provide such a variation. Such a variation can be referred to as providing texture to the combined SRS and textile layer. Incorporating such a variation can provide an improvement in the performance of the motion control system.

It will be appreciated by the skilled person that a given wearable item may comprise multiple layers of strain-rate sensitive material, each of which are attached to the wearable item by a different one of the above listed means. Thus, for example, a wearable item according to embodiments of the present disclosure may comprise a first layer of strain-rate sensitive material adhered to the textile layer and a second layer of strain-rate sensitive material woven into the textile layer. In embodiments, the at least one layer of strain-rate sensitive material is combined into the textile layer by one or more of woven, non-woven and knitted processes. In embodiments, a single layer of strain-rate sensitive material may be attached to more than one textile layer (for example, such that the layer of strain-rate sensitive material acts to bond the two textile layers together).

In embodiments, the strain-rate sensitive material is attached to the inside of the garment (i.e. to a surface of the fabric which, in use, faces the user's body). In such embodiments, the strain-rate sensitive material may be attached such that, in use, the strain-rate sensitive material is adjacent to (for example, in direct contact with) the user's skin. In embodiments, the strain-rate sensitive material is attached to the outside of the garment (i.e. to a surface of the fabric which, in use, faces away from the user's body). In embodiments, the strain-rate sensitive material is sandwiched between two layers of fabric, such that the strain-rate sensitive material is contained within the garment. In embodiments, the strain-rate sensitive material is not attached to fabric, but is instead free-floating within the fabric (for example, held in a pocket within the fabric).

In embodiments the motion control system comprises first and second layers of strain-rate sensitive material. In such embodiments, it may be that the first and second layers of strain-rate sensitive material are positioned adjacent to one other (for example, such that the first and second layers of strain-rate sensitive material are in direct contact with one another). In alternative embodiments, the first layer of strain-rate sensitive substance is attached to an opposite surface of the fabric to that of the second layer of strain rate sensitive substance. In such embodiments, it may be that the fabric is sandwiched between the first and second layers of strain-rate sensitive material.

In embodiments, the at least one layer of strain-rate sensitive material comprises a continuous sheet. In embodiments, the at least one layer of strain-rate sensitive material comprises a plurality of planar motion controlling cells. In embodiments, the plurality of motion controlling cells (and thereby also the strain-rate sensitive substance) form geometrical anisotropic patterns. It may be that the geometrical anisotropic patterns are determined by use of density mapping to map out areas of high strain of a body part for which the motion control system is configured to constrain motion. Embodiments incorporating motion controlling cells in geometrical anisotropic patterns can allow specific directional strains of the soft tissue/muscle to be targeted and reduced. Forming the motion controlling cells in geometrical anisotropic patterns can provide targeted control of motion, such that undesirable movements are constrained without impeding the physical activity of the user. In such embodiments, the strain-rate sensitive substance may provide increased control of motion in a first direction compared to a second. It will be appreciated that the directionality of the control of motion is determined by the shape of the geometrical patterns. In embodiments, at least one of the plurality of planar motion controlling cells comprises one or more of the following geometries: diagonal lines, vertical lines, horizontal lines, curved lines, squares, diamonds, triangles, hexagons, and auxetic polygons.

In embodiments, the motion controlling cells comprise one or more locking-patterns. In embodiments, a locking-pattern comprises a spring element (for example, in the form of a chevron). In such embodiments, when the locking pattern is pulled, the chevron opens up to approximate a straight line. In embodiments where the motion controlling cells form lines (for example, horizontal or vertical lines), it may be that one or more locking-patterns are arranged on the vertical or horizontal lines, such that the motion controlling cell deviates from the line geometry to form the locking pattern. In embodiments, one or more locking patterns are positioned away from the ends of the motion controlling cell, such that the cell can be considered to deviate from the line geometry to form the locking pattern before subsequently continuing the line geometry. Alternatively or additionally, one or more locking patterns may be positioned at an end of a motion controlling cell.

In embodiments, at least one (for example, all) of the plurality of planar motion controlling cells comprises a geometry determined by a surface tessellation process. In embodiments, the surface tessellation process comprises a Voronoi tessellation process. Performing a Voronoi tessellation process may comprise generating a plurality of notional seed points in pseudo-random locations on the surface of the wearable item. The surface is then divided into regions defined according to which of the notional seed points is the closest. Thus, any given location on the surface will sit within a region associated with the nearest of the notional seed points. The regions define an array of dissimilar irregular polygons that together cover the entirety of the surface of the wearable item. In embodiments, each of those regions comprises a motion controlling cell.

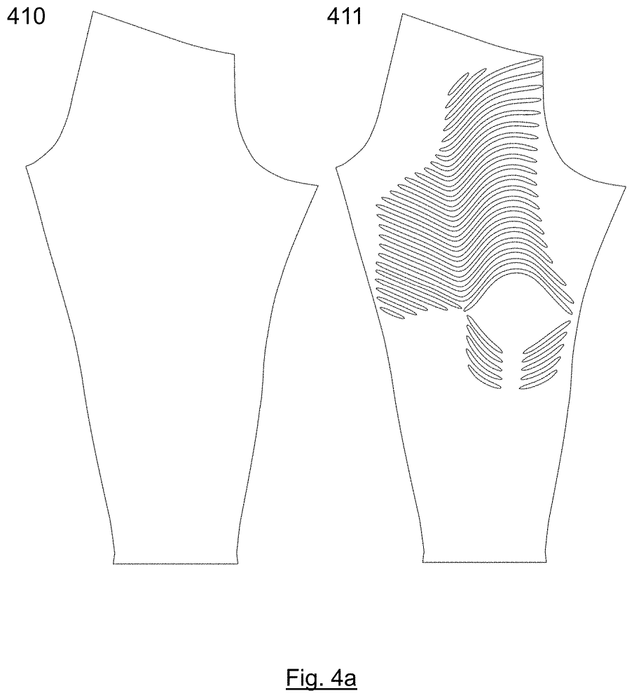

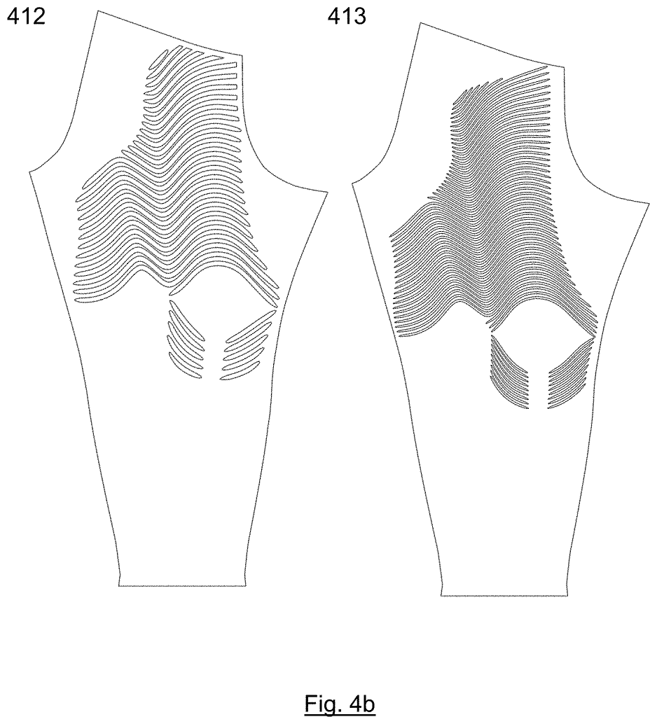

FIG. 4 shows a number of configurations of motion control systems for shorts according to embodiments of the present disclosure. A first example pair of shorts 41 comprises SRS material in a solid laminate layer. The remaining example pairs of shorts each comprise a layer of SRS material having a plurality of planar motion controlling cells. A second example pair of shorts 42 comprises motion controlling cells in the form of diagonal lines. A third example pair of shorts 43 comprises motion controlling cells in the form of vertical lines. A fourth example pair of shorts 44 comprises motion controlling cells in the form of horizontal lines. A fifth example pair of shorts 45 comprises motion controlling cells in the form of a square grid pattern. A sixth example pair of shorts 46 comprises motion controlling cells in the form of a diamond grid pattern. A seventh example pair of shorts 47 comprises motion controlling cells in the form of a tessellating triangle pattern. An eighth example pair of shorts 48 comprises motion controlling cells in the form of a tessellating hexagonal grid pattern. A ninth example pair of shorts 49 comprises motion controlling cells in the form of a Voronoi grid pattern.

FIGS. 4a and 4b show a number of configurations of motion control systems for tights (or leggings) according to embodiments of the present disclosure. A first example pair of tights 410 comprises SRS material in a solid laminate layer. The remaining example pairs of tights each comprise a layer of SRS material having a plurality of planar motion controlling cells. A second example pair of tights 411 comprises motion controlling cells in the form of a relatively low number of thick strips/waves. A third example pair of tights 412 comprises motion controlling cells in the form of a relatively medium number of thinner strips/waves. A fourth example pair of tights 413 comprises motion controlling cells in the form of a relatively high number of still thinner strips/waves.

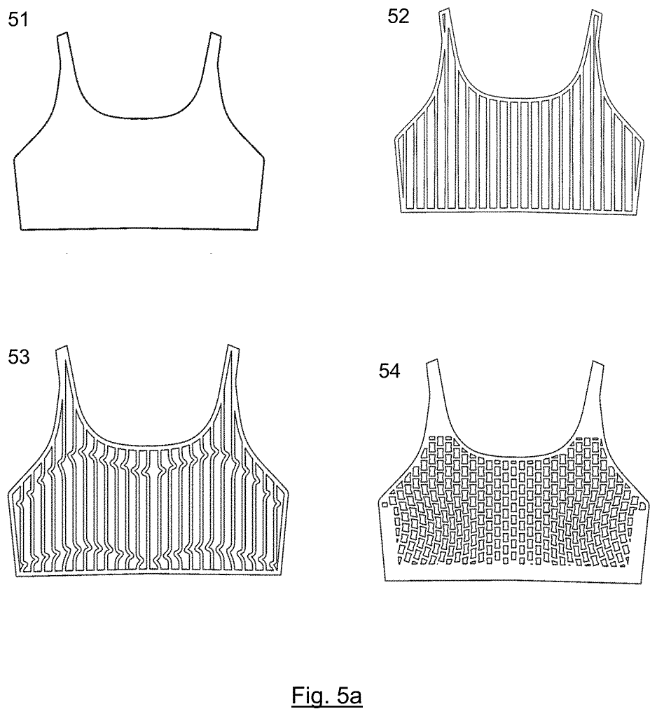

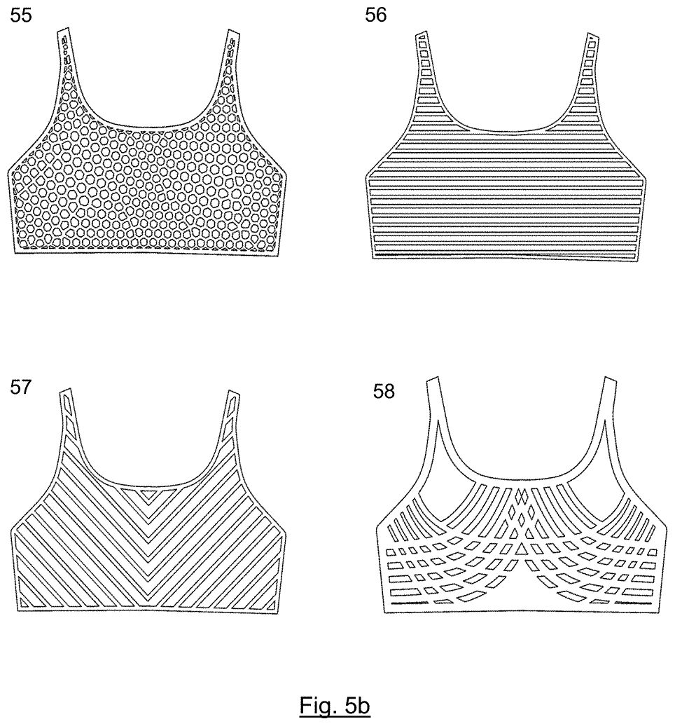

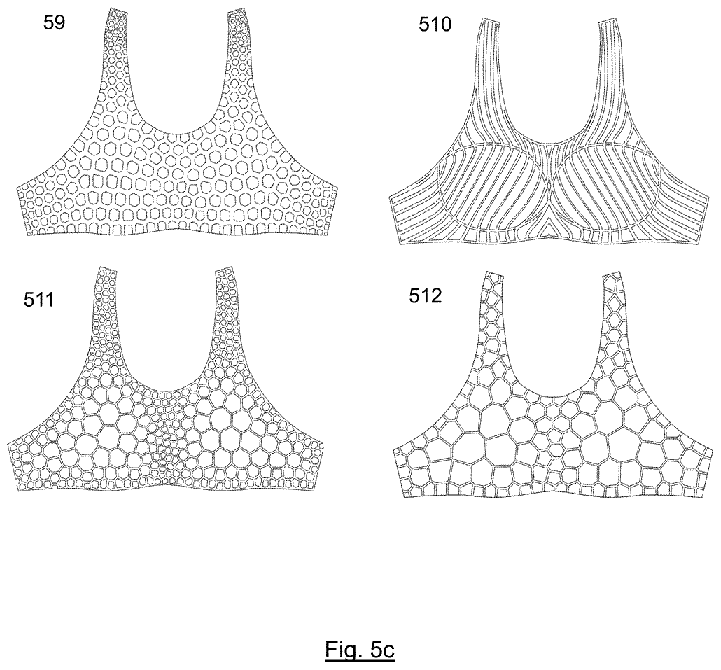

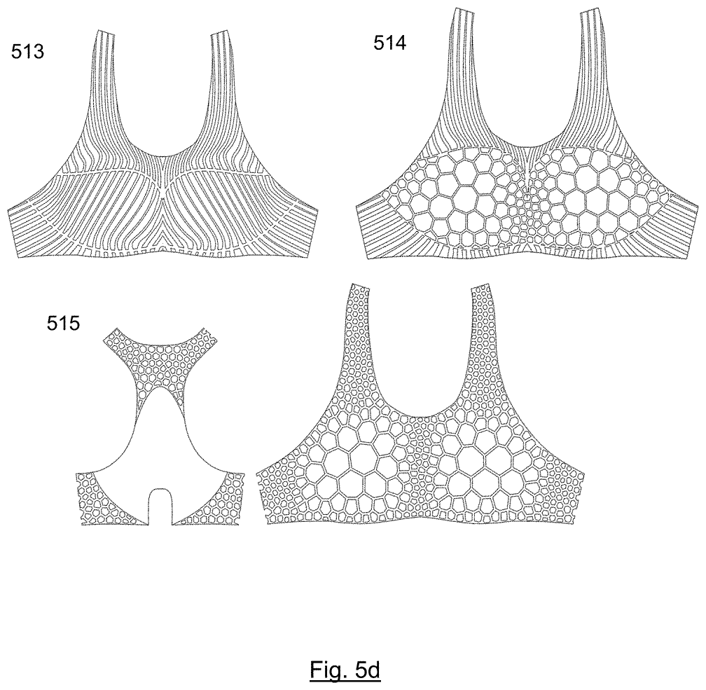

FIGS. 5a, 5b, 5c, and 5d show a number of configurations of motion control systems for sports bras according to embodiments of the present disclosure. A first example sports bra 51 comprises SRS material in a solid laminate layer. The remaining example sports bras each comprise a layer of SRS material having a plurality of planar motion controlling cells. A second example sports bra 52 comprises motion controlling cells in the form of vertical lines. A third example sports bra 53 comprises motion controlling cells in the form of vertical lines including locking patterns. A fourth example sports bra 54 comprises motion controlling cells in the form of an array of auxetic polygons. A fifth example sports bra 55 comprises motion controlling cells in the form of a Voronoi grid. A sixth example sports bra 56 comprises motion controlling cells in the form of horizontal lines. A seventh example sports bra 57 comprises motion controlling cells in the form of diagonal lines. An eighth example sports bra 58 comprises motion controlling cells in the form of curved lines. A ninth example sports bra 59 comprises motion controlling cells in the form of a Voronoi grid. A tenth example sports bra 510 comprises motion controlling cells in the form of zoned thin lines. An eleventh example sports bra 511 comprises motion controlling cells in the form of a zoned 300 μm Voronoi grid. A twelfth example sports bra 512 comprises motion controlling cells in the form of zoned 150 μm Voronoi grid. A thirteenth example sports bra 513 comprises motion controlling cells in the form of zoned lines. A fourteenth example sports bra 514 comprises motion controlling cells in the form of both zoned lines and a zoned Voronoi grid. A fifteenth example sports bra 515 comprises motion controlling cells in the form of an extended Voronoi grid which also includes a view of the part of the bra that is adjacent to the wearer's back during use.

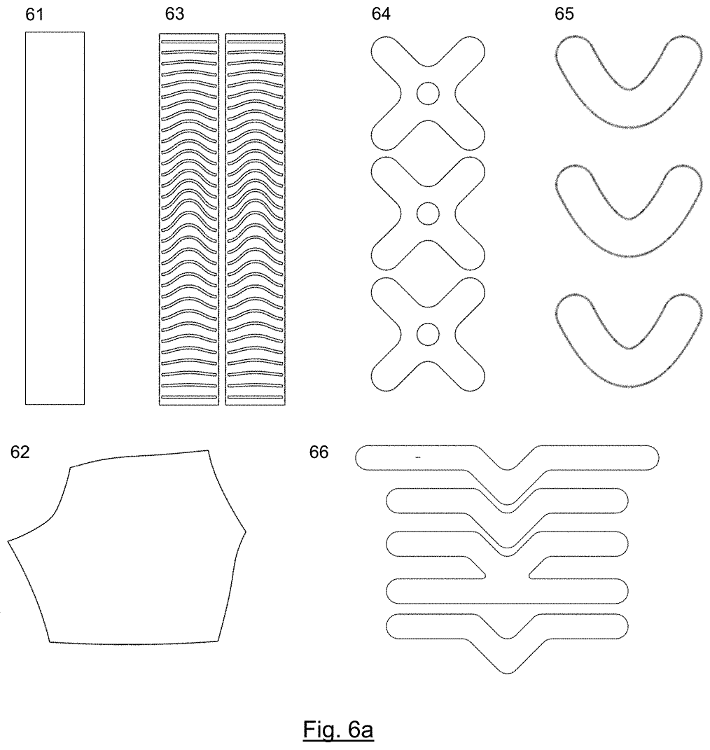

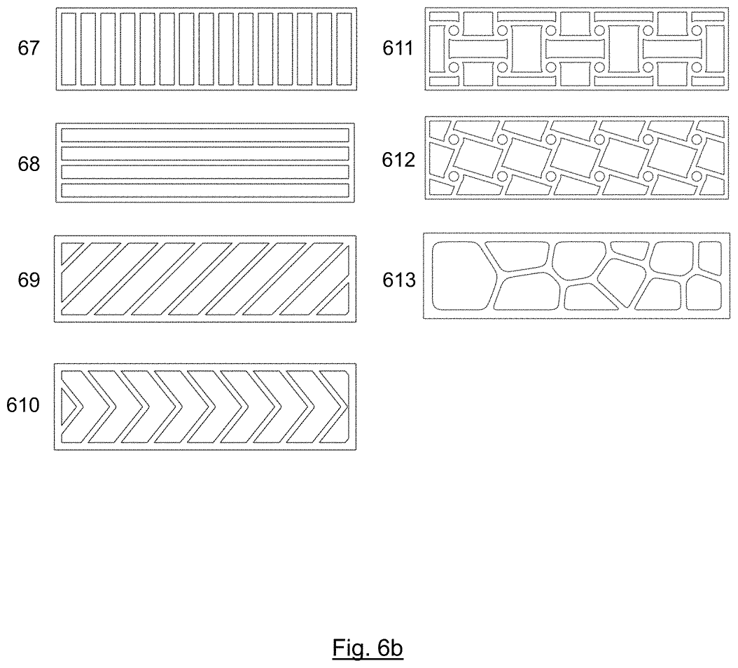

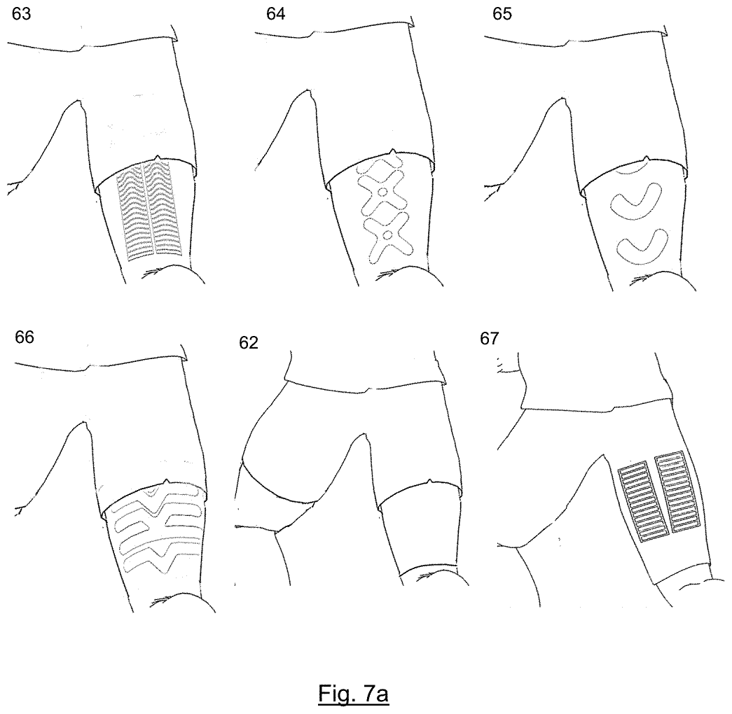

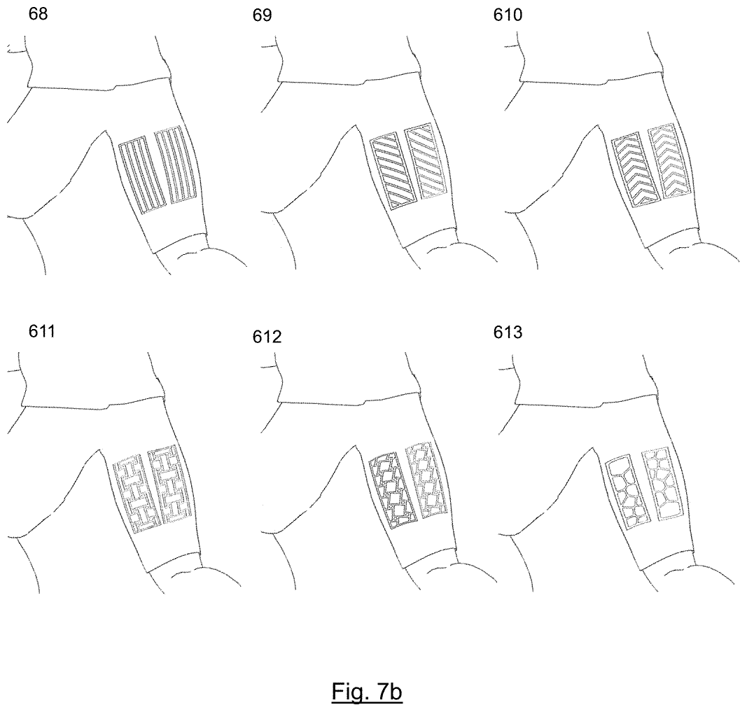

FIGS. 6a and 6b show a number of configurations of motion control systems for kinesiology tape (or ‘motion control tape’) according to embodiments of the present disclosure. FIGS. 7a and 7b show perspective views of each of the kinesiology tape configurations of FIGS. 6a and 6b applied to the thigh of a wearer. A first example kinesiology tape 61 and a second example kinesiology tape 62 each comprise SRS material in a solid laminate layer. A third example kinesiology tape 63 comprises SRS material in a wave pattern with varying gradient. The remaining example kinesiology tapes each comprise a layer of SRS material having a plurality of planar motion controlling cells. A fourth example kinesiology tape 64 comprises motion controlling cells in the form of a diagonal cross pattern. A fifth example kinesiology tape 65 comprises motion controlling cells in the form of a V-shaped pattern. A sixth example kinesiology tape 66 comprises motion controlling cells in the form of an indented lines pattern. The indented lines pattern comprises a series of parallel horizontal lines, each with a V-shaped indent. A seventh example kinesiology tape 67 comprises motion controlling cells in the form of horizontal lines. An eighth example kinesiology tape 68 comprises motion controlling cells in the form of vertical lines. A ninth example kinesiology tape 69 comprises motion controlling cells in the form of diagonal lines. A tenth example kinesiology tape 610 comprises motion controlling cells in the form of a chevron pattern. An eleventh example kinesiology tape 611 comprises motion controlling cells in the form of a chiral pattern. A twelfth example kinesiology tape 612 comprises motion controlling cells in the form of an angled chiral pattern. A thirteenth example kinesiology tape 613 comprises motion controlling cells in the form of a Voronoi grid.

In embodiments, the plurality of planar motion controlling cells comprises a first subset of motion controlling cells having a first geometry and a second, different subset of motion controlling cells having a second, different geometry. In such embodiments, it may be that the motion controlling cells in the first subset have different motion control properties from motion controlling cells in the second subset. In embodiments, the motion controlling cells in the first subset are located in a first zone of the wearable item and motion controlling cells in the second subset are located in a second, different zone of the wearable item. Such embodiments can provide wearable items having regions with different mechanical properties. For example, different regions may be configured to provide differing levels of support, or to resist motion in different directions. Thus, a wearable item according to such embodiments will respond differently depending on the direction and speed with which it is stretched. Providing a wearable item with multiple regions having different motion control characteristics can enable “problem” areas of the soft tissue with more undesirable motion to be targeted with increased damping whilst providing greater flexibility (and therefore comfort) in non-“problem” areas.

In embodiments, the motion control system comprises first and second layers of strain-rate sensitive material configured to control motion from movement of one or more body parts of the user. In such embodiments, it may be that the textile layer is sandwiched in-between the first and second layers of strain-rate sensitive material. In embodiments, the motion control system comprises a further textile layer. In such embodiments, it may be that the at least one layer of strain-rate sensitive material layer is sandwiched in-between the textile layer and the further textile layer.

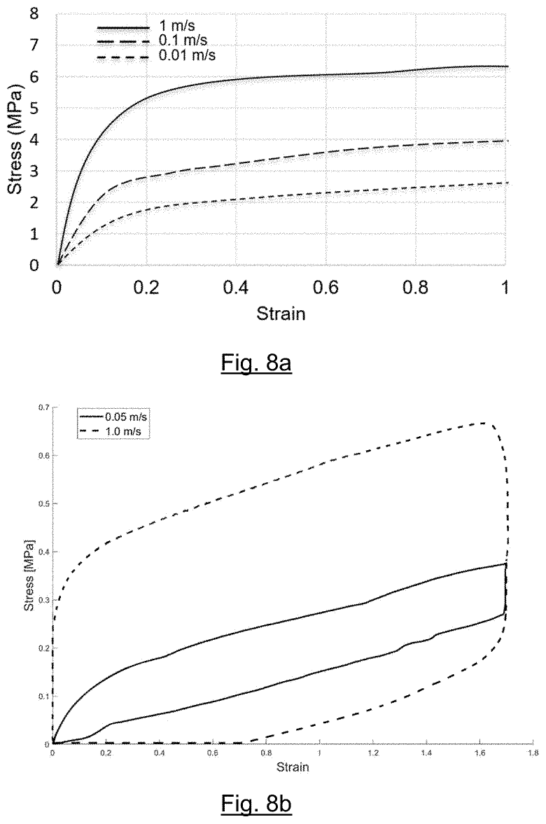

FIG. 8a shows stress-strain curves at different movement speeds (1 m/s, 0.1 m/s, and 0.01 m/s) for an example SRS material according to embodiments of the present disclosure, with an original sample size of 300 mm long and 60 mm wide. It can be seen that the mechanical properties of the material are strain-rate dependent. The higher the rate of strain applied to the substance, the stiffer the material's response to the deformation is. The figure shows the response of the strain-rate sensitive material at three different tensile loadings. The stiffness of the material (indicated by the gradient of the curve) between 0 and 0.5 strain increases drastically with increased rate of deformation. This means at low strain-rates the material is flexible (facilitating donning and doffing) and at higher strain-rates is significantly stiffer (enhancing the performance of the wearable item).

FIG. 8b shows tensile hysteresis curves at low and high strain-rates for an example SRS material according to embodiments of the present disclosure, with an original sample size of 300 mm long and 21 mm wide. The area within the hysteresis loop (which corresponds to the amount of energy absorbed) is larger at the increased strain rate. The damping or energy dissipation properties also increase with quicker motions. Thus, energy control and/or dissipation increase with strain rate.

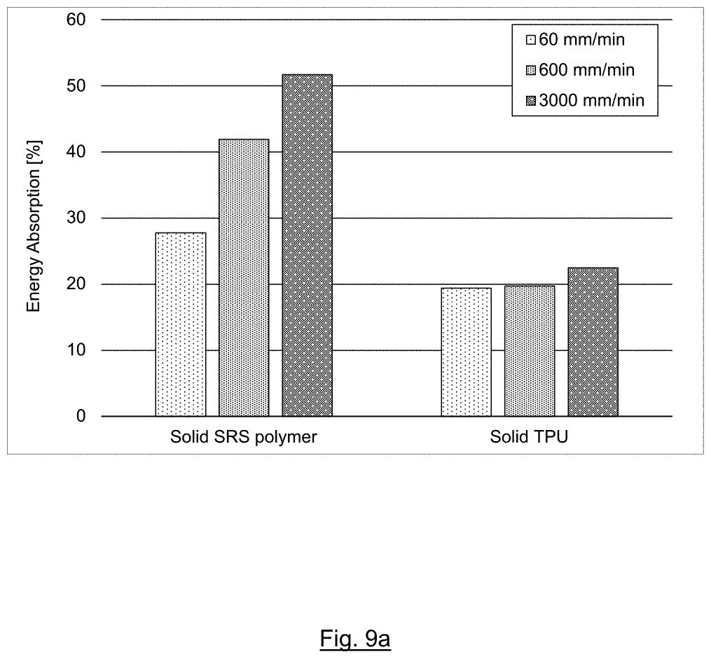

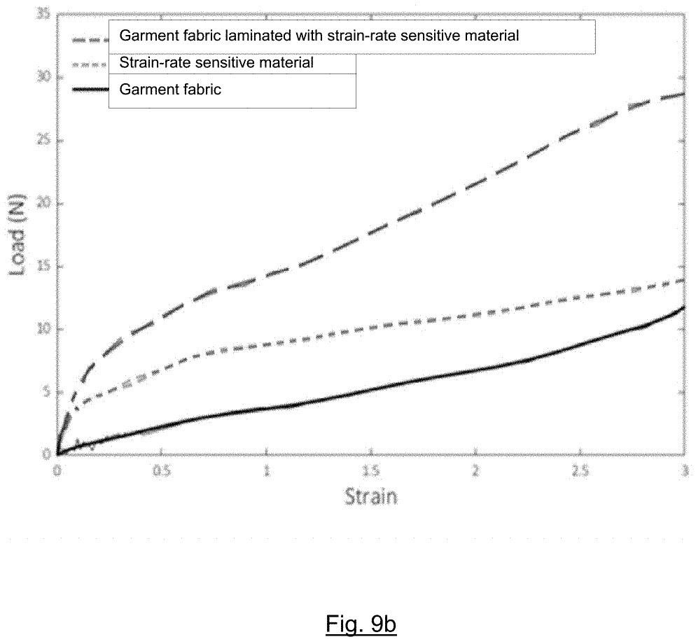

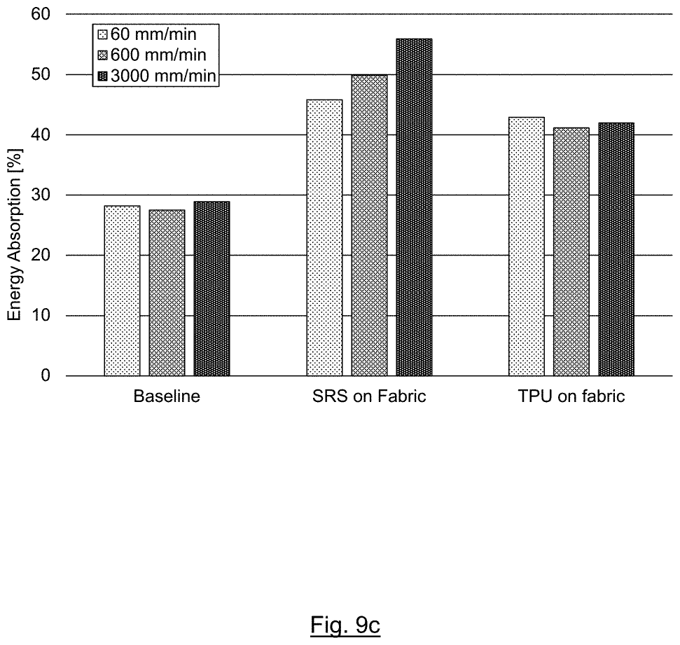

FIG. 9a shows a chart of the energy absorption of strain rate sensitive substance according to embodiments of the present disclosure and a thermoplastic polyurethane (TPU) material. The chart shows that the strain rate sensitive substance provides increased energy absorption at higher strain rates. By contrast the TPU material provides relatively constant energy absorption at all strain rates. Providing a garment with increased energy absorption at higher strain rates according to embodiments allows for improved motion control whilst also facilitating easier donning and doffing of the garment (low strain-rates).

FIG. 9b shows a load strain curve of an example wearable item laminated with a strain rate sensitive substance according to embodiments of the present disclosure. The example wearable item was stretched at a rate of 0.24 m/s, from an original sample size of 300 mm long by 60 mm wide. In embodiments, the wearable item comprises strain-rate sensitive material laminated onto a base garment textile. FIG. 9b illustrates the mechanical properties of an example baseline garment fabric alone, an example strain-rate sensitive material alone, and the strain-rate sensitive material laminated onto the base garment textile (referred to as a “composite” wearable item). The baseline garment fabric exhibits a substantially linear relationship between tensile load and applied strain. This linear relationship exists independently of strain rate. The composite wearable item requires higher loads to achieve any given strain (and thus is stiffer). In particular, the stiffness of the composite wearable item (corresponding to the gradient of the curve) between 0 and 0.5 strain is increased. Thus, the composite wearable item provides stiffer garment response/support. Laminating strain-rate sensitive material onto the base garment textile can therefore be seen to enhance the mechanical properties of the wearable item, increasing the stiffness of the wearable item. It can also be seen that the stiffness is improved in the composite wearable item compared to the strain-rate sensitive substance alone.

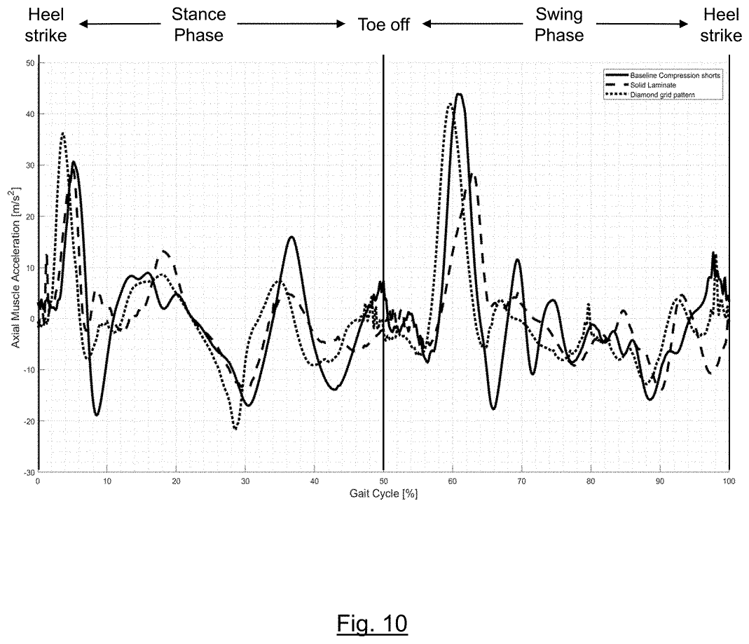

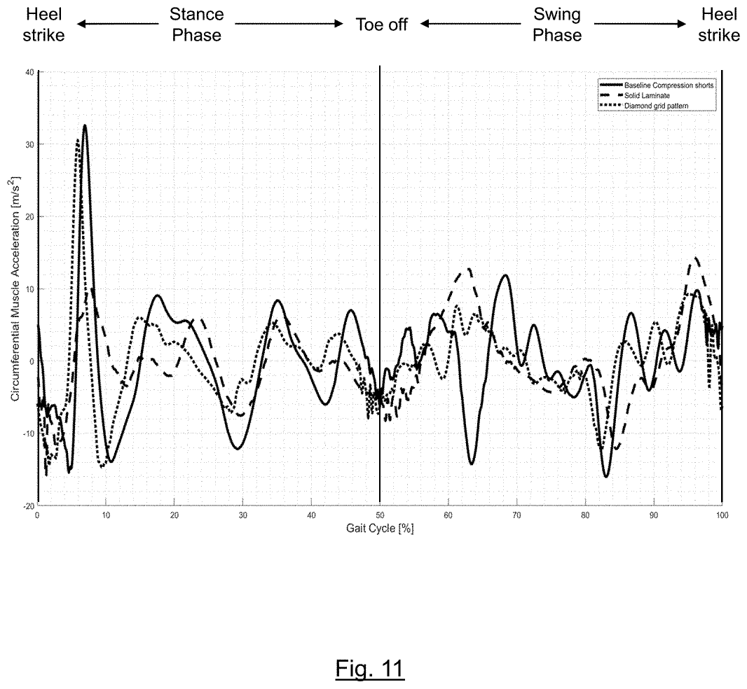

FIG. 9c shows a chart of energy absorption of strain rate sensitive substance laminated onto fabric according to embodiments of the present disclosure, TPU laminated onto fabric, and the baseline fabric alone. The chart shows that the baseline fabric provides relatively constant energy absorption at all strain rates. The TPU laminated onto fabric also provides relatively constant energy absorption at all strain rates, but provides more energy absorption than the baseline fabric. The strain rate sensitive substance laminated onto fabric provides increased energy absorption at higher strain rates. Thus, the SRS material provides increased energy absorption at the increased strain rate both in isolation and when laminated onto a fabric substrate. FIG. 10 shows a graph of the axial acceleration of a user's hamstring muscle when wearing compressions shorts of the prior art (referred to as the “baseline compression shorts”) and when wearing shorts according to embodiments of the present disclosure.

FIG. 11 shows a graph of the circumferential acceleration of the user's hamstring muscle.

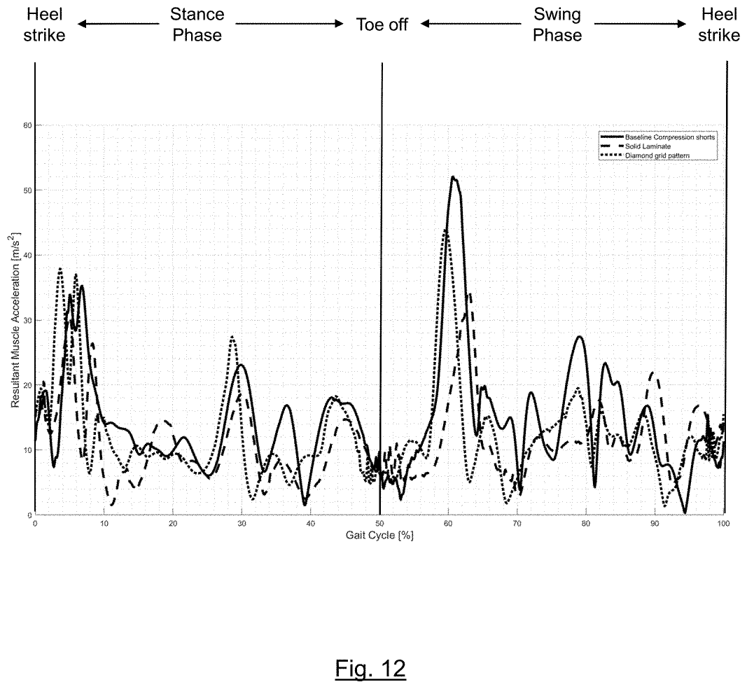

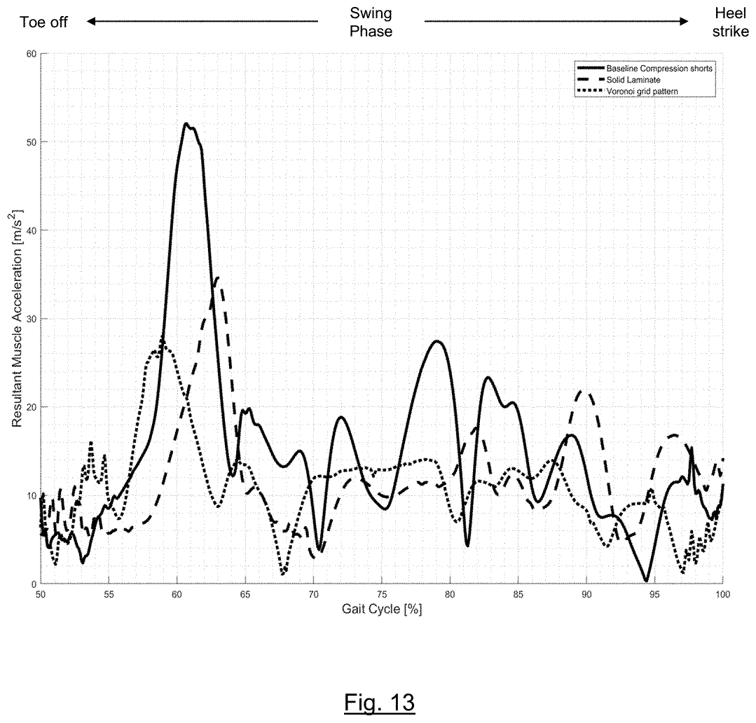

FIG. 12 shows a graph of the resultant acceleration magnitude of the user's hamstring muscle. In each of FIGS. 10 to 12, the baseline compression shorts are indicated by a solid line, shorts having a solid laminate layer of strain-sensitive material are indicated by a dashed line, and shorts having a diamond grid patterned layer of strain-sensitive material are indicated by a dotted line. For both the shorts having solid laminate SRS material and diamond grid patterned SRS material, the amplitude of the muscle acceleration over the gait cycle was reduced compared to both non-compressive and compressive shorts of the prior art.

FIG. 13 demonstrates the resultant accelerations of the hamstring muscle during the swing phase (between toe off and the next heel strike) of a user wearing the compression shorts garment. Baseline compression shorts are indicated by a solid line, shorts having a solid laminate layer of strain-sensitive material are indicated by a dashed line, and shorts having a Voronoi grid patterned layer of strain-sensitive material are indicated by a dotted line. For the shorts with the SRS material in the Voronoi grid pattern, the resultant peak and RMS accelerations are reduced compared to the baseline compressions shorts.

It is thought that a body close wearable item incorporating a strain rate sensitive material can enhance the efficiency of an athlete's performance by controlling the energy of the run. During the swing phase of the gait cycle, the kinetic energy of the muscle is used to position the leg from the toe off to the next heel strike stage. During this phase, high frequency muscle motion (“wobble”) in circumferential and radial directions results in the inefficient use of energy and therefore inefficient energy management by the athlete. Assuming kinetic energy is proportional to the square of the velocity and considering the peak velocity of the axial muscle acceleration, then the shorts with a solid laminate layer of SRS material are preserving 24% more energy compared to the baseline running shorts. On that same basis, the shorts with a Voronoi grid patterned layer of SRS material are preserving 14% more energy compared to the baseline running shorts. This saved energy can then be transformed to useful kinetic energy during the stance stage of the next gait cycle and thereby improve the athlete's performance overall.

As previously discussed, wearable items according to embodiments of the present disclosure can include planar motion controlling cells of SRS material of a number of different geometries. Table 1 below shows for each of a number of different geometries of motion controlling cells: (a) axial RMS acceleration [m/s²], (b) improvement in axial acceleration [%], (c) circumferential RMS acceleration [m/s²], (d) improvement in circumferential acceleration [%], (e) radial RMS acceleration [m/s²], (f) improvement in radial acceleration [%], (g) resultant RMS acceleration [m/s2], and (h) improvement in resultant acceleration [%] and (i) area coverage of the SRS laminate [cm²].

TABLE 1

| Sample Name | (a) | (b) | (c) | (d) | (e) | (f) | (g) | (h) | (i) |
|---|---|---|---|---|---|---|---|---|---|
| No Compression shorts | 13.04 | 0% | 10.43 | 0% | 11.24 | 0% | 20.42 | 0% | — |
| Baseline Compression | 13.33 | −2% | 9.30 | 11% | 11.61 | −3% | 19.99 | 2% | — |
| shorts | | | | | | | | | |
| Solid Laminate | 9.08 | 30% | 6.06 | 42% | 8.62 | 23% | 13.90 | 32% | 1428.73 |
| Diagonal Line | 10.24 | 21% | 6.31 | 40% | 9.33 | 17% | 15.23 | 25% | 573.90 |
| Vertical Lines | 9.78 | 25% | 7.59 | 27% | 9.12 | 19% | 15.51 | 24% | 594.77 |
| Horizontal lines | 10.14 | 22% | 6.52 | 37% | 8.98 | 20% | 15.11 | 26% | 563.24 |
| Square grid pattern | 10.46 | 20% | 6.36 | 39% | 8.74 | 22% | 15.05 | 26% | 696.76 |
| Diamond grid pattern | 10.65 | 18% | 6.59 | 37% | 9.34 | 17% | 15.63 | 23% | 573.90 |
| Triangles grid pattern | 10.52 | 19% | 6.99 | 33% | 9.15 | 19% | 15.65 | 23% | 638.30 |
| Hexagonal grid pattern | 10.73 | 18% | 6.58 | 37% | 8.15 | 28% | 15.03 | 26% | 620.02 |
| Voronoi grid pattern | 9.52 | 27% | 6.24 | 40% | 8.78 | 22% | 14.46 | 29% | 488.54 |

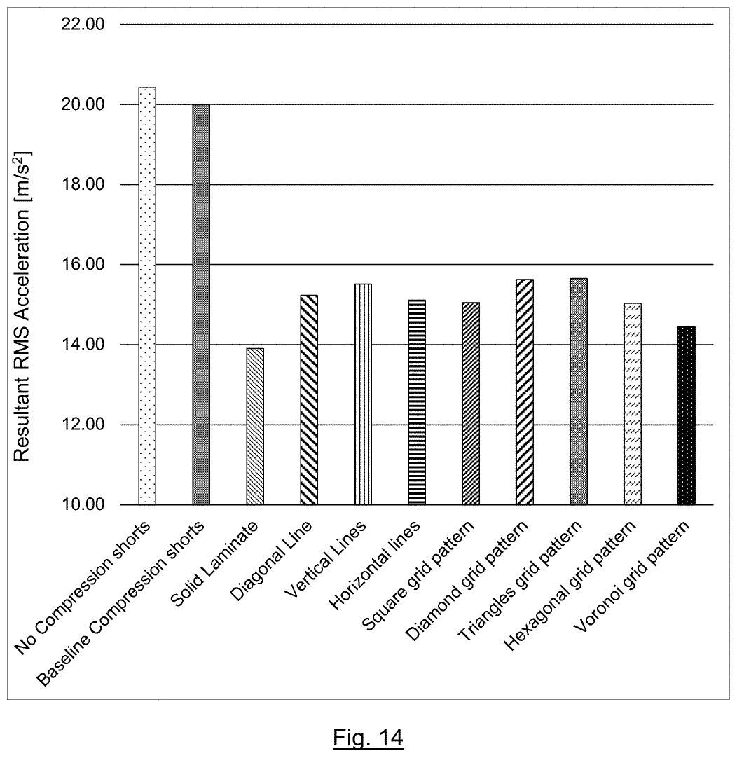

Table 1 shows that a greater quantity of strain rate sensitive substance yields a greater reduction in the resultant muscle acceleration. In particular, the solid laminate geometry shows the greatest improvement in the resultant RMS acceleration of 32% compared to no compression shorts. FIG. 14 shows a chart of the resultant RMS acceleration of a user's hamstring muscle when wearing shorts having different geometries of planar motion controlling cells according to embodiments of the present disclosure (as listed in Table 1 above).

Table 2 below shows the effect of different amounts of muscle mass on the effectiveness of shorts according to the present disclosure. Table 2 shows (a) resultant RMS acceleration [m/s²], and (b) improvement in resultant acceleration [%] for two users of differing muscle mass.

TABLE 2

| | More muscle mass | | Less muscle mass | |
|---|---|---|---|---|
| Sample Name | (a) | (b) | (a) | (b) |
| No Compression shorts | 19.47 | 0% | 21.36 | 0% |
| Solid Laminate | 12.83 | 34% | 14.98 | 30% |
| Diagonal Line | 14.04 | 28% | 16.42 | 23% |
| Vertical Lines | 13.37 | 31% | 17.65 | 17% |
| Horizontal lines | 13.18 | 32% | 17.05 | 20% |
| Square grid pattern | 14.11 | 28% | 16.00 | 25% |
| Diamond grid pattern | 14.51 | 25% | 16.75 | 22% |
| Triangles grid pattern | 14.71 | 24% | 16.59 | 22% |
| Hexagonal grid pattern | 13.52 | 31% | 16.54 | 23% |
| Voronoi grid pattern | 12.12 | 38% | 16.79 | 21% |

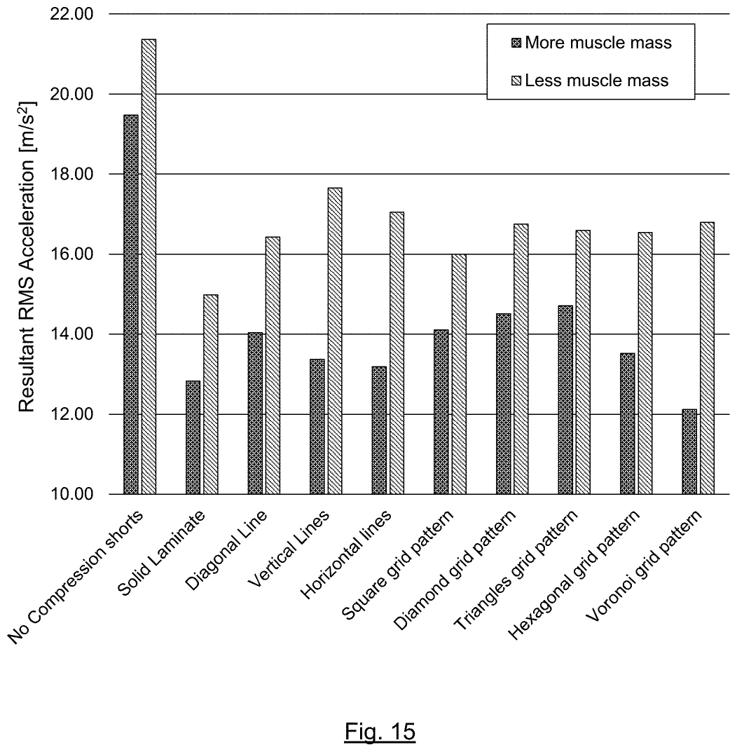

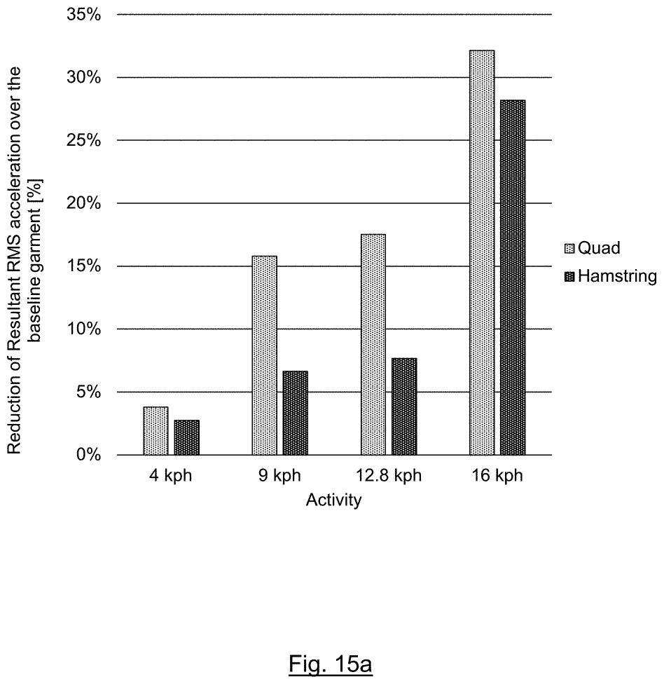

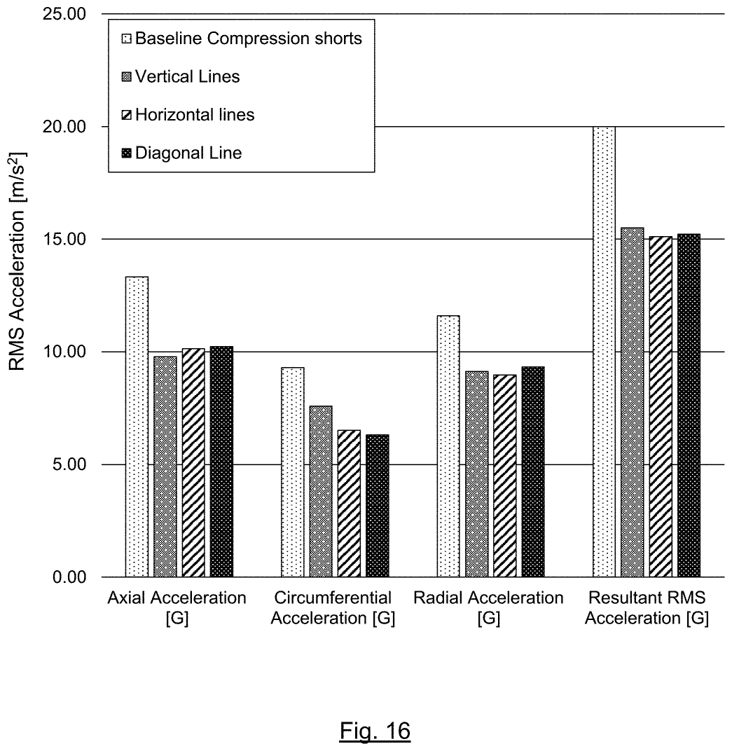

Table 2 shows that, the more muscle mass the user has, the greater the reduction in acceleration provided by shorts according to the present disclosure. For each of the geometries, the resultant RMS acceleration was further reduced for the user with more muscle mass than for the user with less. This is because a greater quantity of muscle mass creates more inertia when running, causing higher deformation and therefore higher stiffness of the strain rate sensitive substance, resulting in more limited muscle movement. FIG. 15 shows a chart of the resultant RMS acceleration of the hamstring muscles of two users with different levels of muscle mass when wearing shorts having different geometries of planar motion controlling cells according to embodiments of the present disclosure (as listed in Table 2 above).

FIG. 15a shows a chart of the reduction (compared to baseline shorts) of the resultant RMS acceleration of the quadriceps and hamstring of a user wearing compression shorts according to embodiments of the present disclosure whilst running at various speeds. FIG. 15a shows that, for all running speeds, the motion control system delivered a greater reduction in RMS resultant acceleration on the quadriceps than on the hamstring. It is believed that this difference is due to the greater mass of the quadriceps compared to the hamstring. A person's quadriceps are typically approximately 2.4 times the mass of their hamstring.

FIG. 16 shows a chart comparing accelerations of a user's hamstring when wearing compression shorts of the prior art to when wearing shorts according to embodiments of the present disclosure. In particular, the graph compares baseline compression shorts with shorts having vertical, horizontal, and diagonal lines of SRS material. The graph shows that specific geometries of motion controlling cell can be used to tackle muscle accelerations in a specific direction (i.e. axial, radial, or circumferential) whilst having minimal effect on the accelerations in the other directions. Directional accelerations are generally improved more by geometries in which the pattern is aligned with the direction of muscle acceleration. Thus, a vertical lines pattern has a greater effect on the axial acceleration compared to a horizontal lines pattern. Similarly, a horizontal lines pattern has the greatest reduction in RMS muscle acceleration in the circumferential direction.

When torque is applied to a tensile element, the direction of the principal stress in the element is at 45 degrees to the direction of the axial motion. FIG. 16 shows that diagonal lines yield a greater reduction in the RMS circumferential muscle acceleration compared to vertical or horizontal lines. Controlling circumferential movement could be important for reducing the risks of hamstring injury. Limiting such movement will reduce the torsional load on the tendon as there will be less muscle twisting movement.

Table 3 below shows for each of the baseline compression shorts and shorts having solid laminate and Voronoi patterned motion controlling cells: (a) axial RMS acceleration [m/s²], (b) improvement in axial acceleration [%], (c) circumferential RMS acceleration [m/s²], (d) improvement in circumferential acceleration [%], (e) radial RMS acceleration [m/s²], (f) improvement in radial acceleration [%], (g) resultant RMS acceleration [m/s²], (h) improvement in resultant RMS acceleration [%], and (i) area [cm²] of SRS material.

TABLE 3

| Sample Name | (a) | (b) | (c) | (d) | (e) | (f) | (g) | (h) | (i) |
|---|---|---|---|---|---|---|---|---|---|
| Baseline | 13.33 | 0% | 9.30 | 0% | 11.61 | 0% | 19.99 | 0% | — |
| Compression | | | | | | | | | |
| shorts | | | | | | | | | |
| Solid Laminate | 9.08 | 32% | 6.06 | 35% | 8.62 | 26% | 13.90 | 30% | 1428.73 |
| Voronoi grid | 9.52 | 29% | 6.24 | 33% | 8.78 | 24% | 14.46 | 28% | 488.54 |
| pattern | | | | | | | | | |

Table 3 shows that some geometries of motion controlling cells can provide significant improvements in performance whilst also enabling a reduction in the weight of the motion control system. This solid laminate provides 30% reduction in resultant RMS acceleration compared to the baseline shorts with a coverage area of 1428.73 cm², while the Voronoi grid pattern provides 28% reduction in resultant RMS acceleration and has just 488.54 cm² (66% less area coverage). The solid laminate is also less comfortable to wear, don and doff than the Voronoi pattern due to the additional coverage area of SRS material. This table demonstrates that, even with a significant reduction in the coverage area of SRS material, the motion control system can still perform almost as well as full coverage solid laminate, whilst also being easier to don and doff and more comfortable to wear.

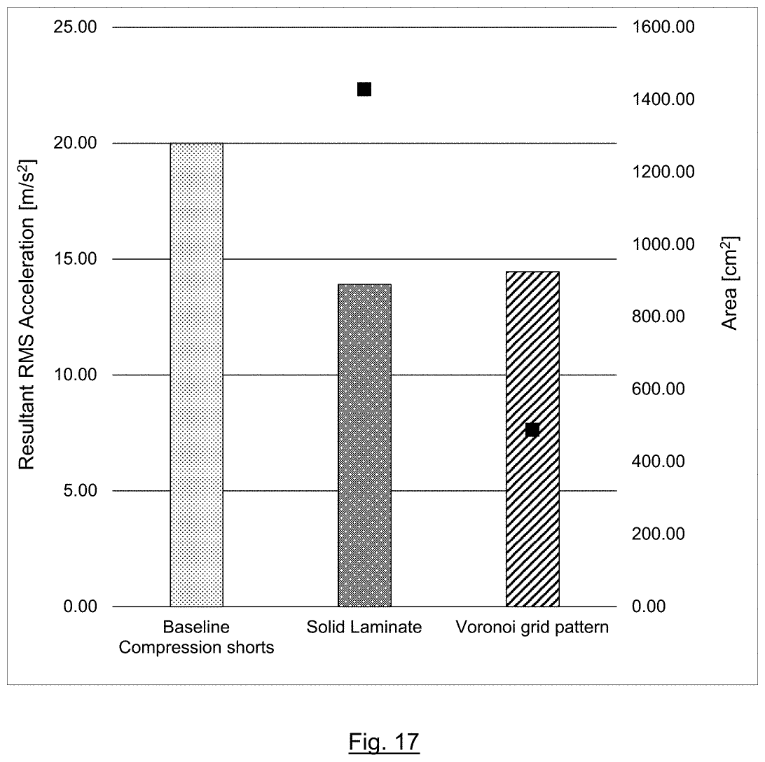

FIG. 17 shows a chart of the resultant RMS acceleration of a user's hamstring when wearing shorts according to embodiments of the present disclosure, along with the total area of strain rate sensitive material in each pair of shorts (as listed in Table 3).

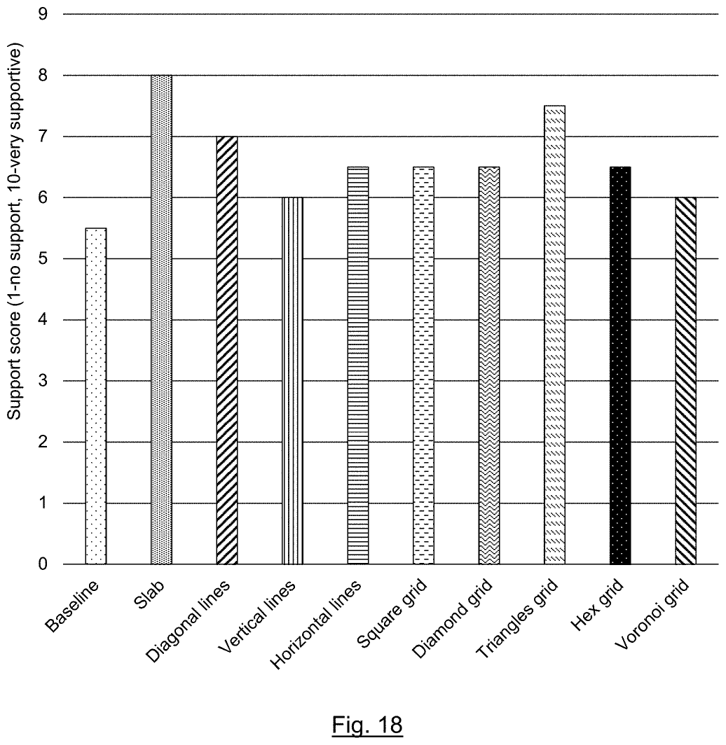

FIG. 18 shows a chart of the perceived support given by shorts according to embodiments of the present disclosure. The chart shows that perceived support increases as the amount of SRS material is increased. This is shown to be the case for all of the tested geometries of motion controlling cells.

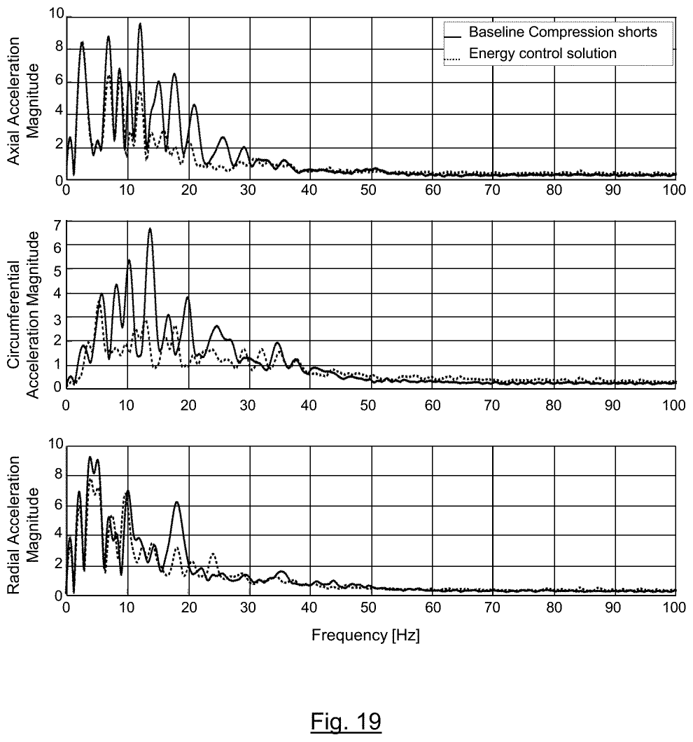

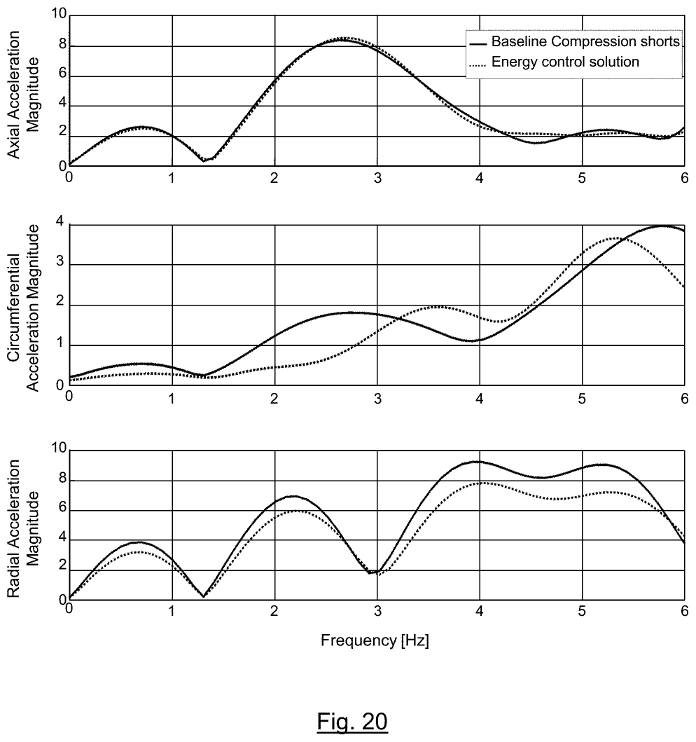

FIG. 19 shows a graph of the magnitude with respect to frequency of the acceleration of a user's hamstring when wearing compression shorts of the prior art and shorts according to embodiments of the present disclosure. The graph shows that, for shorts according to the present disclosure, the magnitude of muscle accelerations in the frequency region 1-6 Hz (main body running frequency) are relatively unaffected. However, a significant reduction in magnitude is observed at higher frequencies (10-40 Hz), which correspond to muscle “wobble” as the athlete runs. This is beneficial, as normal running frequencies are unimpeded and only the detrimental muscle “wobble” frequencies are reduced.

FIG. 20 shows a graph of the magnitude with respect to frequency of the acceleration of the user's hamstring at the main body running frequencies. It can be seen that there is little difference between acceleration magnitudes for shorts according to the present disclosure and for baseline compression shorts at these frequencies.

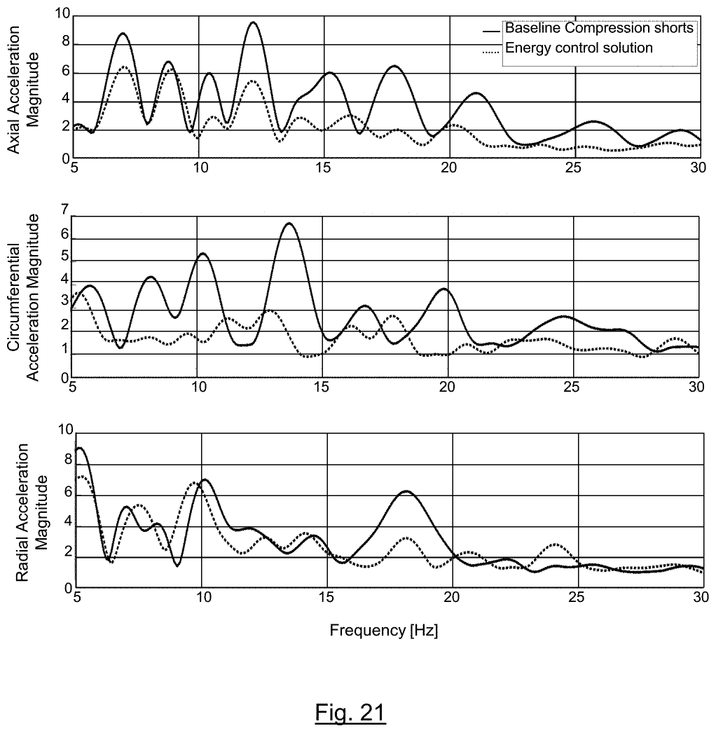

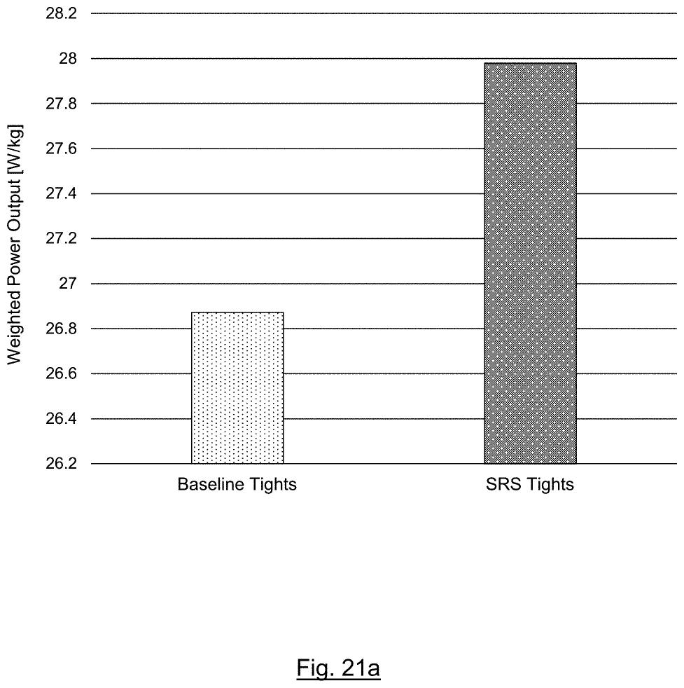

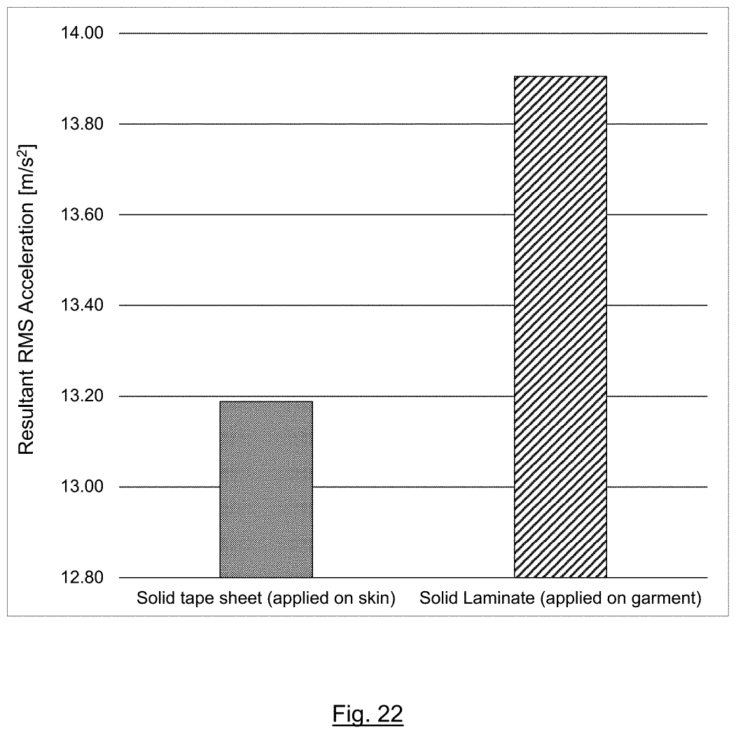

FIG. 21 shows a graph of the acceleration magnitude with respect to frequency of the acceleration of the user's hamstring at the higher muscle “wobble” frequencies. It can be seen that at these frequencies there is significant reduction in acceleration magnitudes for the shorts according to the present disclosure compared to the baseline compression shorts.

FIG. 21a shows a chart of the weighted power output of a user wearing motion control tights according to embodiments of the present disclosure. The weighted power output was measured by having subjects undertake ten counter-weight jumps whilst standing on a force plate. FIG. 21a shows that subjects had, on average, a 4.12% increase in power output when wearing tights having a motion control system compared to the baseline tights. It is believed that this is due to the improved control of circumferential and radial motion of the subjects' muscles provided by the SRS material enabling greater efficiency. In particular, it is believed that the improved control during the landing phase of the exercise preserves energy for use in the subsequent jump.

In embodiments, the wearable item comprises kinesiology tape. Table 4 shows the resultant RMS acceleration of a hamstring muscle of a user of kinesiology tape according to the present disclosure when the tape is applied directly onto the user's skin and when it is applied onto a garment compared to the no compression shorts baseline.

TABLE 4

| Sample Name | Resultant RMS Acceleration [m/s²] |
|---|---|
| No Compression shorts | 21.36 |
| Solid tape sheet (applied on skin) | 13.19 |
| Solid Laminate (applied on garment) | 13.90 |

Table 4 shows that the effect of the SRS material is increased when the tape is applied directly to the skin, as there is no slippage and friction between a textile and the skin, which could lead to less efficient energy control. When the SRS material was applied as a solid laminate onto a garment, the resultant muscle RMS acceleration improvement over the baseline decreased to 35% compared to the 38% improvement of tape applied directly to skin. Hence, a greater improvement can be achieved with the direct application of strain-rate sensitive substance to the skin. FIG. 22 shows a chart of the resultant RMS acceleration of a muscle of a user of kinesiology tape according to embodiments of the present disclosure when applied directly onto the skin and when applied onto a garment (as listed in Table 4).

Table 5 shows a comparison of prior art kinesiology tape (henceforth referred to as “baseline tape”) with a number of configurations of kinesiology tape according to the present disclosure. Table 5 shows: (a) axial RMS acceleration [m/s²], (b) improvement in axial acceleration [%], (c) circumferential RMS acceleration [m/s²], (d) improvement in circumferential acceleration [%], (e) radial RMS acceleration [m/s²], (f) improvement in radial acceleration [%], (g) resultant RMS acceleration [m/s²], and (h) improvement in resultant RMS acceleration [%].

TABLE 5

| Sample | | | | | | | | |
|---|---|---|---|---|---|---|---|---|
| Name | (a) | (b) | (c) | (d) | (e) | (f) | (g) | (h) |
| Baseline | 11.89 | 0% | 15.68 | 0% | 9.88 | 0% | 22.02 | 0% |
| tape | | | | | | | | |
| Solid tape | 7.81 | 34% | 6.52 | 58% | 8.39 | 15% | 13.19 | 40% |
| sheet | | | | | | | | |
| Gradated | 11.66 | 2% | 12.86 | 18% | 9.23 | 7% | 19.66 | 11% |
| wave tape | | | | | | | | |
| Diagonal | 10.54 | 11% | 12.54 | 20% | 8.80 | 11% | 18.60 | 16% |
| cross tape | | | | | | | | |
| V tape | 10.31 | 13% | 10.89 | 31% | 9.25 | 6% | 17.62 | 20% |
| Indented | 8.80 | 26% | 7.55 | 52% | 8.97 | 9% | 14.66 | 33% |
| tape | | | | | | | | |

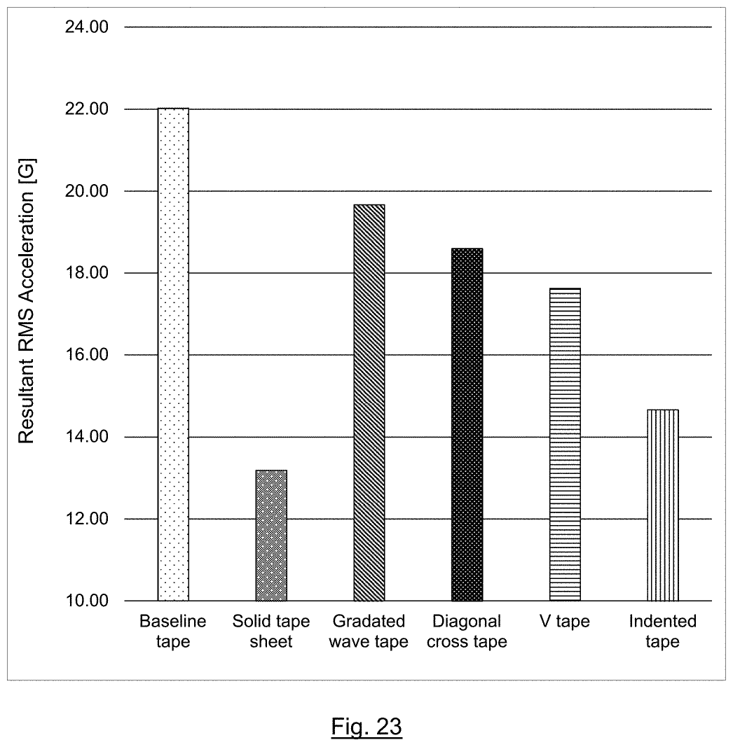

Table 5 shows that the more strain-rate sensitive substance that is used to control the muscle acceleration, the greater the effect on performance. Solid motion control tape wrapped around the circumference of the leg provided the greatest reduction in muscle acceleration (40%) over the baseline kinesiology tape. FIG. 23 shows a chart of the resultant RMS acceleration of a user's muscle when using kinesiology tape according to embodiments of the present disclosure (as listed in Table 5).

Table 6 below shows a comparison of V shaped and indented tape according to embodiments of the present disclosure with baseline tape. Table 6 shows: (a) axial RMS acceleration [m/s²], (b) improvement in axial acceleration [%], (c) circumferential RMS acceleration [m/s²], (d) improvement in circumferential acceleration [%], (e) radial RMS acceleration [m/s²], (f) improvement in radial acceleration [%], (g) resultant RMS acceleration [m/s²], and (h) improvement in resultant RMS acceleration [%].

TABLE 6

| Sample Name | (a) | (b) | (c) | (d) | (e) | (f) | (g) | (h) |
|---|---|---|---|---|---|---|---|---|
| Baseline tape | 11.89 | 0% | 15.68 | 0% | 9.88 | 0% | 22.02 | 0% |
| V tape | 10.31 | 13% | 10.89 | 31% | 9.25 | 6% | 17.62 | 20% |
| Indented tape | 8.80 | 26% | 7.55 | 52% | 8.97 | 9% | 14.66 | 33% |

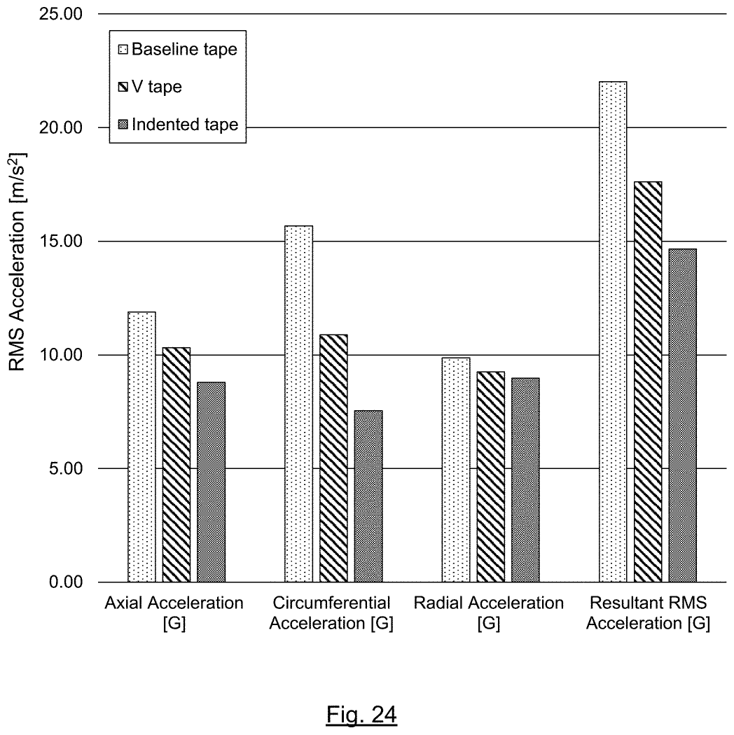

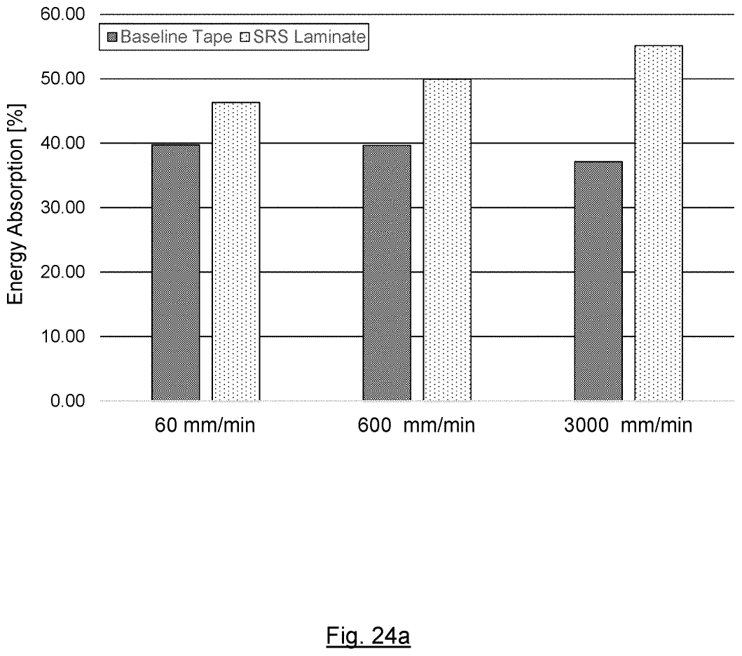

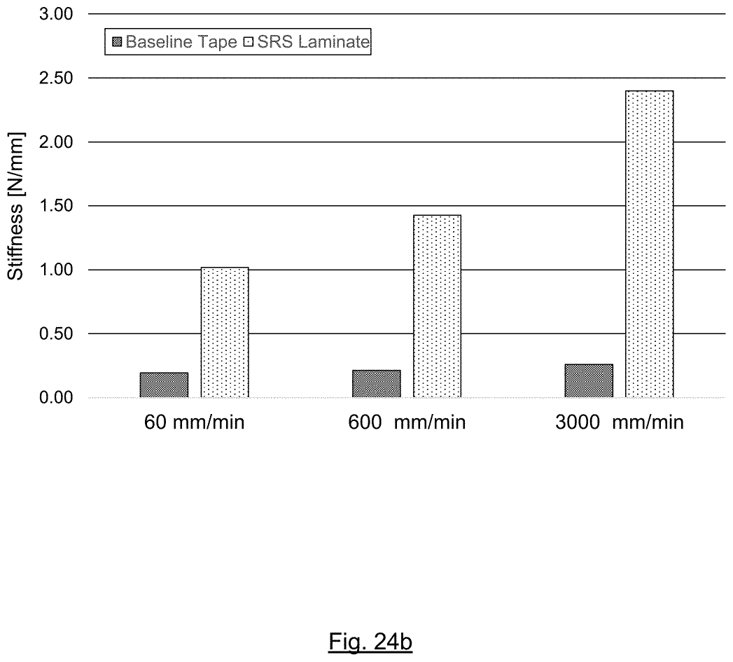

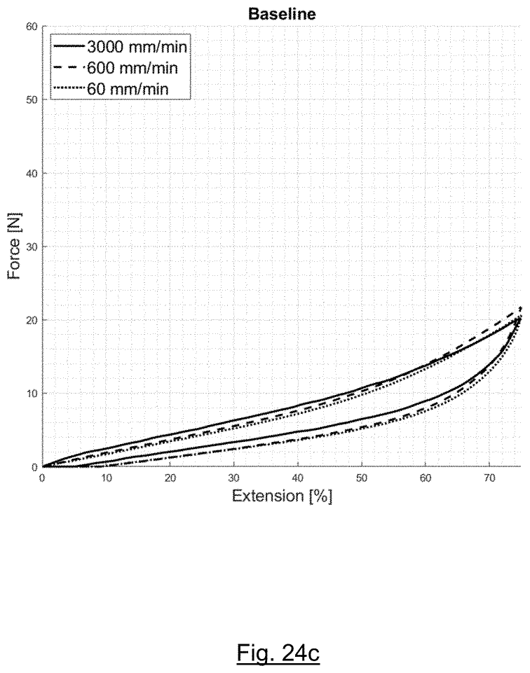

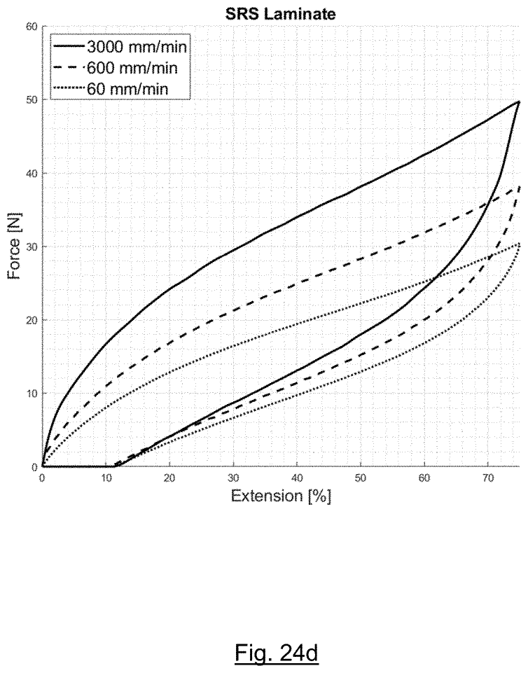

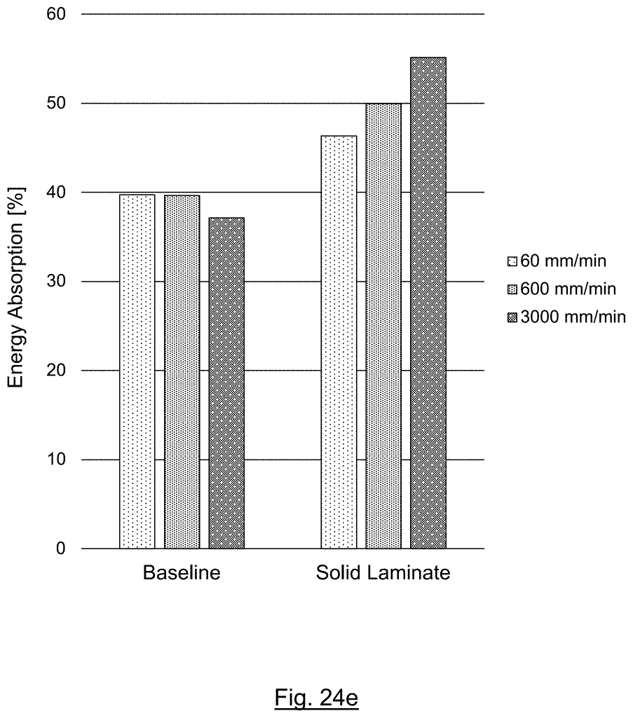

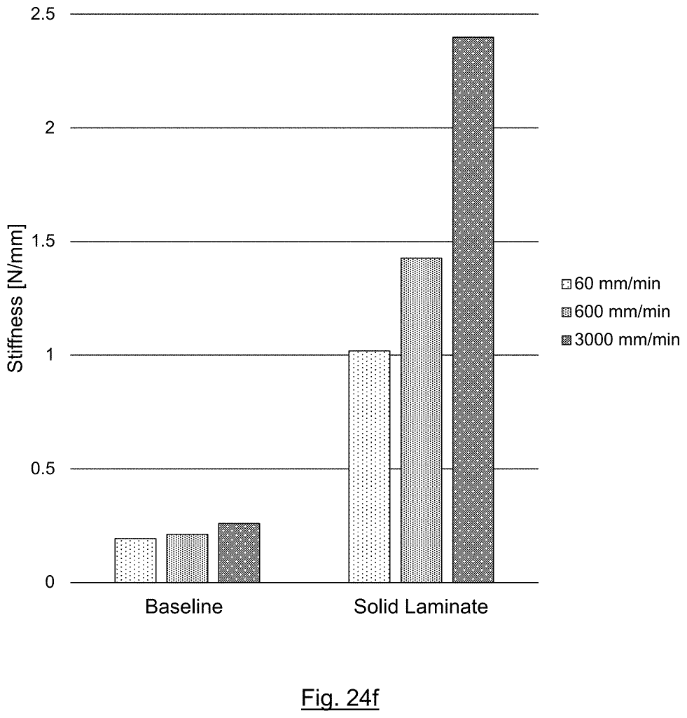

Table 6 shows that both V shaped and indented tape offer significant performance improvements when compared to the baseline tape. This helps to reduce HIS through reduction of circumferential motion. When torque is applied to a tensile element, the direction of the principal stress in the element is at 45 degrees to the direction of the axial motion. Hence, reducing the stresses in the plane at 45 degrees to the muscle direction is thought to provide a greater reduction effect in the probability of hamstring injury. A significant effect was achieved with the 45-degree cross pattern (arrow patch) applied directly to the athlete skin. FIG. 24 shows a chart of the accelerations of a user's muscle when using kinesiology tape according to embodiments of the present disclosure (as listed in Table 6).

FIG. 24a shows a chart of the energy absorption of motion control tape according to embodiments of the present disclosure at a number of stretch speeds. FIG. 24a shows that motion control tape having an SRS laminate motion control system provides an increase in energy absorption over baseline motion control tape of 17% at a stretch speed of 60 mm/min, 26% at a stretch speed of 600 mm/min, and 49% at a stretch speed of 3000 mm/min. Thus, motion control tape according to embodiments of the present disclosure not only provides an increase in energy absorption at all stretch speeds, but also provides increasing energy absorption with increasing stretch speed.

FIG. 24b shows a chart of the stiffness of motion control tape according to embodiments of the present disclosure at a number of stretch speeds. FIG. 24b shows that motion control tape having an SRS laminate motion control system provides an increase in stiffness over baseline motion control tape of 427% at a stretch speed of 60mm/min, 571% at a stretch speed of 600 mm/min, and 825% at a stretch speed of 3000 mm/min. Thus, motion control tape according to embodiments of the present disclosure not only provides an increase in stiffness at all stretch speeds, but also provides increasing stiffness with increasing stretch speed.

FIG. 24c shows hysteresis curves of baseline motion control tape at a number of stretch speeds. FIG. 24d shows a hysteresis curve of motion control tape according to embodiments of the present disclosure at those same stretch speeds. In this example, the motion control tape comprises a motion control system having a laminate layer of SRS material. FIGS. 24c shows that the baseline motion control tape exhibits similar properties at all stretch speeds. By contrast, as is shown in FIG. 24d, motion control tape according to embodiments of the present disclosure exhibits increasing stiffness (shown by the gradient of the line between 0 and 10% extension) and energy absorption (which is related to the area bound by the loading and unloading lines) as stretch speed increases.

FIG. 24e shows a chart comparing the energy absorption of motion control tapes of FIGS. 24c and 24d. FIG. 24e shows that the baseline motion control tape demonstrates similar energy absorption at all stretch speeds. By contrast, the motion control tape according to embodiments of the present disclosure demonstrates increased energy absorption at increased strain-rates.

FIG. 24f shows a chart comparing the energy absorption of motion control tapes of FIGS. 24c and 24d. FIG. 24f shows that the baseline motion control tape demonstrates a similar degree of stiffness at all stretch speeds. By contrast, the motion control tape according to embodiments of the present disclosure demonstrates increased stiffness at increased strain-rates.

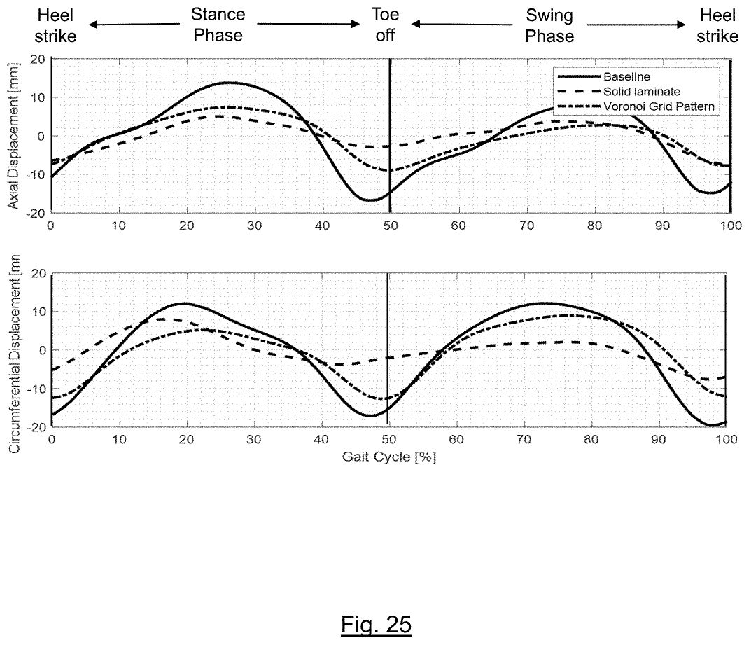

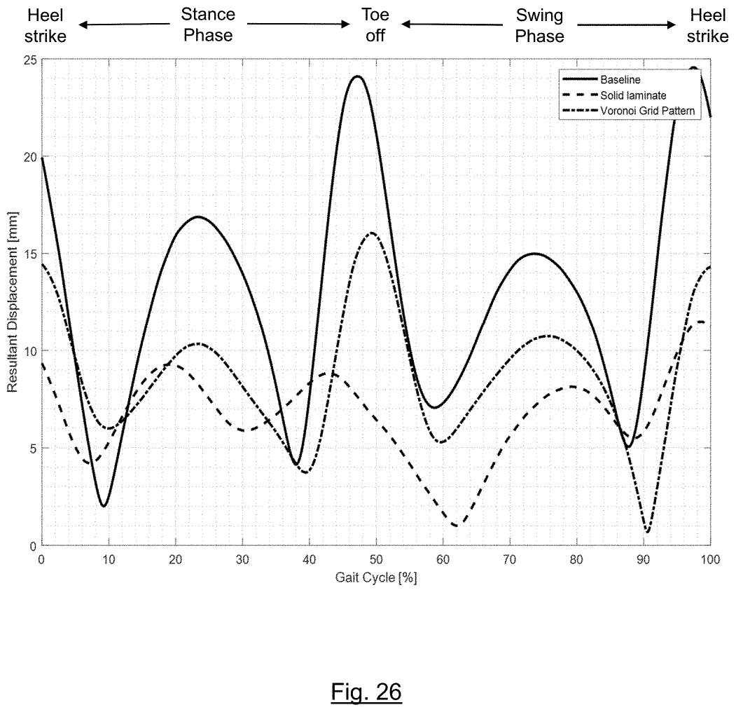

FIG. 25 shows a graph of the axial and circumferential displacement of the breast tissue of a user when wearing a sports bra of the prior art (‘baseline’) and sports bras (solid laminate and Voronoi grid pattern) according to embodiments of the present disclosure. FIG. 26 shows a graph of resultant displacement of the breast tissue of the user. These graphs show that a sports bra incorporating SRS material according to embodiments of the present disclosure can reduce a user's breast displacement. The root mean square value (RMS) of the displacement of the breast relative to the clavicle over the gait cycle was reduced for sports bras according to embodiments of the present disclosure compared to a baseline sports bra. The displacement of the breast in targeted directions is reduced for example walking, jogging, running and star jumping activities.

Table 7 shows a comparison of a number of sports bras having different configurations of motion controlling cells according to embodiments of the present disclosure when worn by a user running at 10 km/hr. Table 7 shows: (a) axial RMS displacement [mm], (b) improvement in axial displacement [%], (c) circumferential RMS displacement [mm], (d) improvement in circumferential displacement [%], and (e) area coverage of the SRS material [cm²].

TABLE 7

| Sample Name | (a) | (b) | (c) | (d) | (e) |
|---|---|---|---|---|---|
| Baseline sports bra | 8.93 | 0.00% | 10.23 | 0.00% | — |
| Solid laminate 600 μm | 3.32 | 62.86% | 3.81 | 62.71% | 570.87 |
| Vertical lines 600 μm | 6.36 | 28.82% | 10.31 | −0.82% | 210.94 |
| Vertical locking 600 μm | 5.84 | 34.65% | 8.90 | 13.01% | 227.38 |
| Zoned auxetic 600 μm | 5.14 | 42.44% | 5.60 | 45.21% | 175.24 |
| Voronoi grid 600 μm | 6.94 | 22.28% | 10.11 | 1.13% | 408.58 |
| Solid laminate 300 μm | 3.89 | 56.47% | 5.26 | 48.59% | 570.87 |
| Vertical lines 300 μm | 6.77 | 24.23% | 9.99 | 2.36% | 210.94 |
| Vertical locking 300 μm | 5.28 | 40.85% | 8.55 | 16.41% | 227.38 |
| Zoned auxetic 300 μm | 5.19 | 41.82% | 9.48 | 7.32% | 175.24 |
| Voronoi grid 300 μm | 4.79 | 46.35% | 6.82 | 33.33% | 408.582 |
| Horizontal lines 300 μm | 6.83 | 23.50% | 9.08 | 11.27% | 245.29 |
| Diagonal lines 300 μm | 6.17 | 30.92% | 10.21 | 0.20% | 218.92 |
| Curved lines 300 μm | 6.57 | 26.45% | 9.18 | 10.21% | 397.34 |

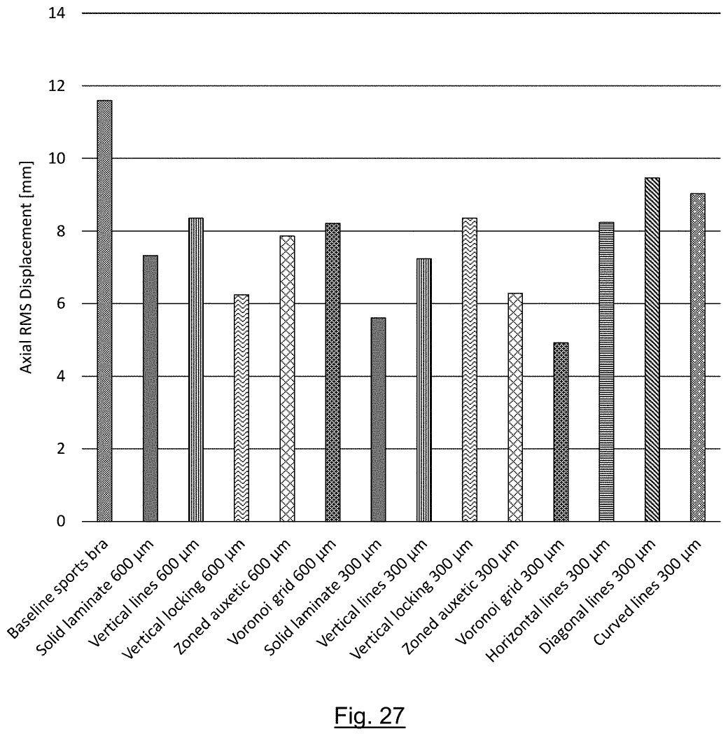

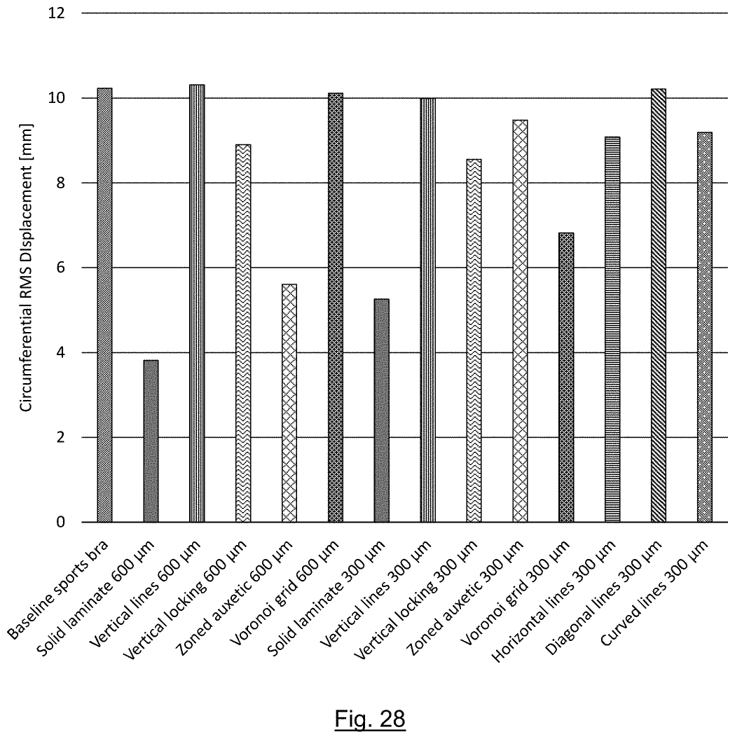

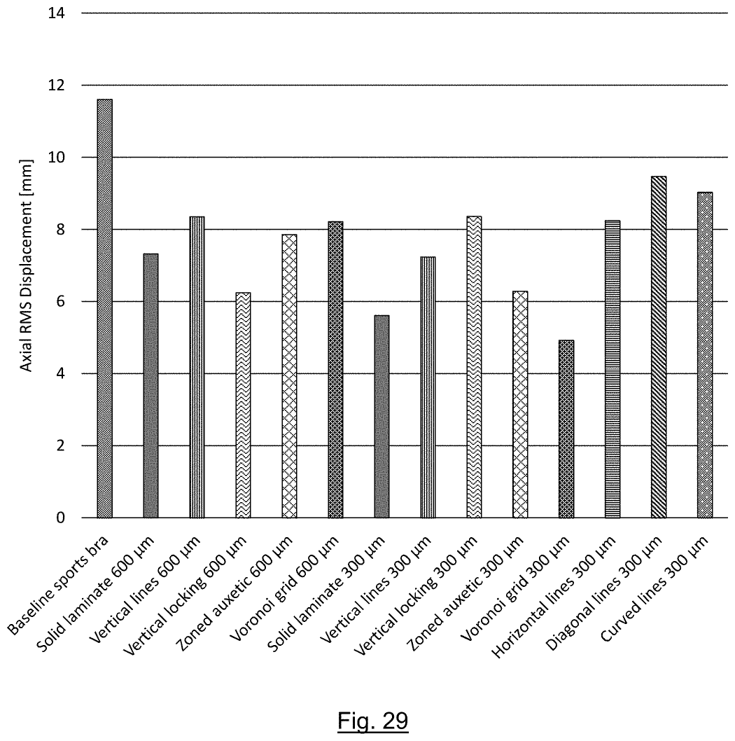

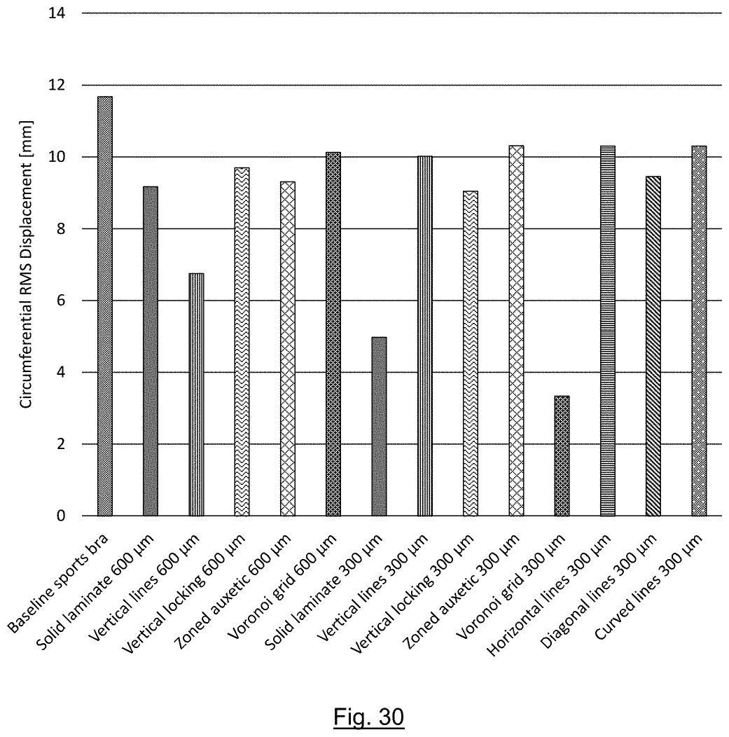

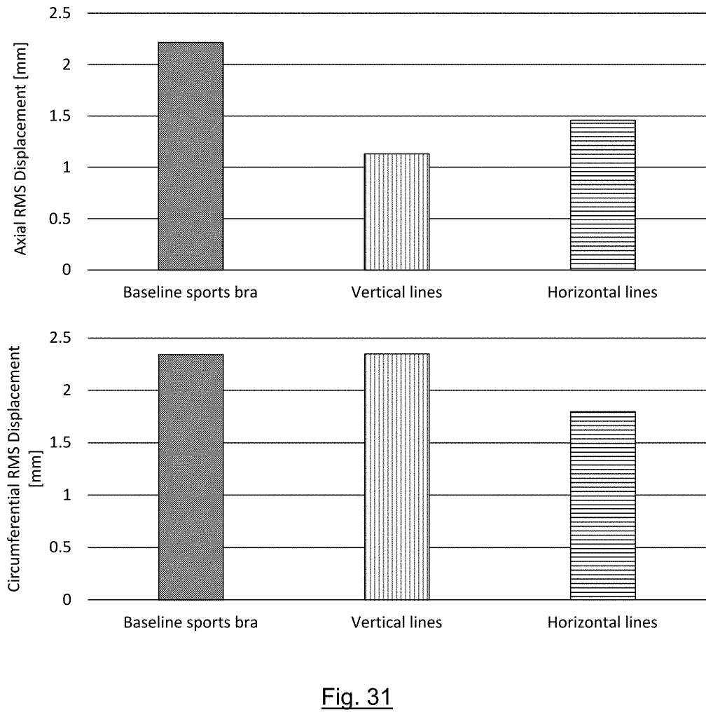

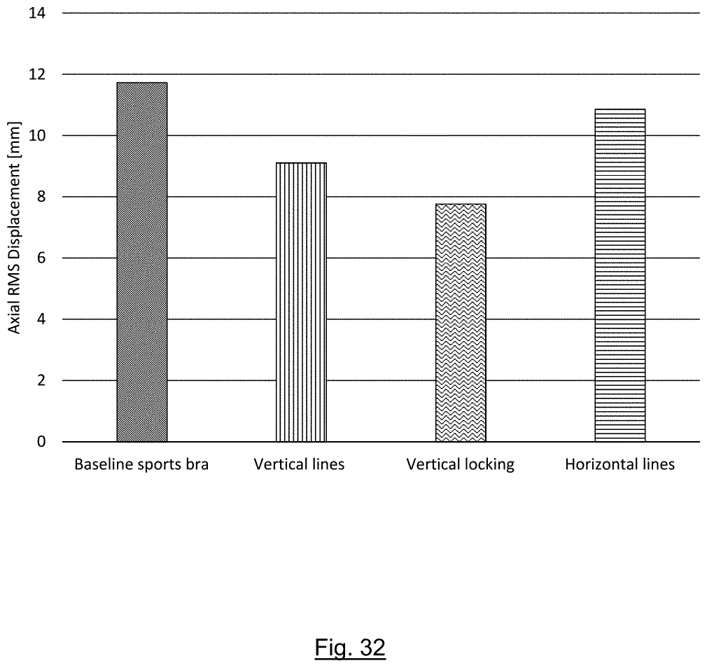

Table 7 shows that a greater area of strain-rate sensitive substance laminated to the sports bra can reduce the displacement (in targeted dimensions) of the breast during running. In particular, the solid laminate 600 micron configuration demonstrated the greatest improvement in both axial and circumferential displacement when jogging at 10 km/hr and also when running at 13 km/hr. FIG. 27 shows a chart of the axial RMS displacement of the breast tissue of a user when wearing sports bras according to embodiments of the present disclosure when running at 10 km/hr (as listed in Table 7). FIG. 28 shows a chart of the circumferential RMS displacement of the breast tissue of the user when running at 10 km/hr (as also listed in Table 7). FIG. 29 shows a chart of the axial RMS displacement of the breast tissue of the user when running at 13 km/hr. FIG. 30 shows a chart of the circumferential RMS displacement of the breast tissue of the user when running at 13 km/hr.

Table 8 shows a comparison of sports bras having motion controlling cells in horizontal and vertical lines patterns according to embodiments of the present disclosure with a sports bra of the prior art (henceforth referred to as the “baseline sports bra”). Table 8 shows: (a) axial RMS displacement [mm], (b) improvement in axial displacement [%], (c) circumferential RMS displacement [mm], and (d) improvement in circumferential displacement [%].

TABLE 8

| Sample Name | (a) | (b) | (c) | (d) |
|---|---|---|---|---|
| Baseline sports bra | 2.21 | — | 2.34 | — |
| Vertical lines | 1.13 | 48% | 2.34 | 0% |
| Horizontal lines | 1.49 | 34% | 1.79 | 23% |

Table 8 shows that, similarly to the shorts of embodiments, vertical geometric features are more effective in controlling the vertical motion of the breast, while horizontal geometric features are better at controlling the circumferential motion. The vertical lines improve the axial displacement by 48% compared to the baseline sports bra, but do not improve the circumferential displacement. Horizontal lines improve the circumferential displacement by 23%. FIG. 31 shows charts of the circumferential and axial RMS displacements of the breast tissue of a user when wearing sports bras according to embodiments of the present disclosure whilst running at 6 km/hr (as listed in Table 8).

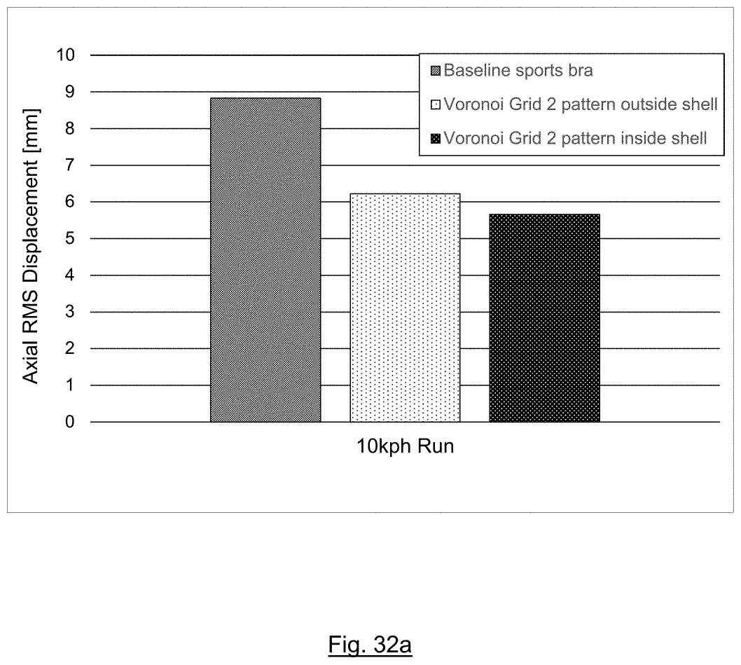

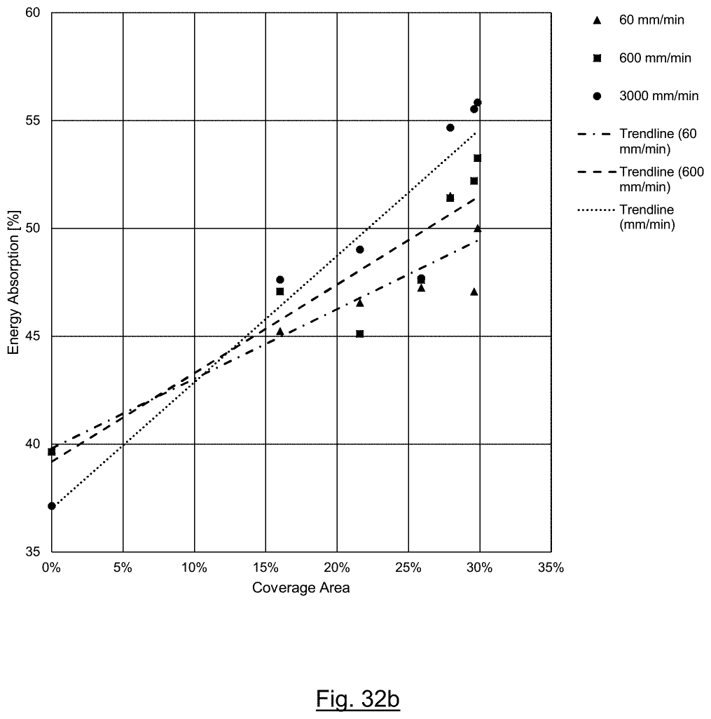

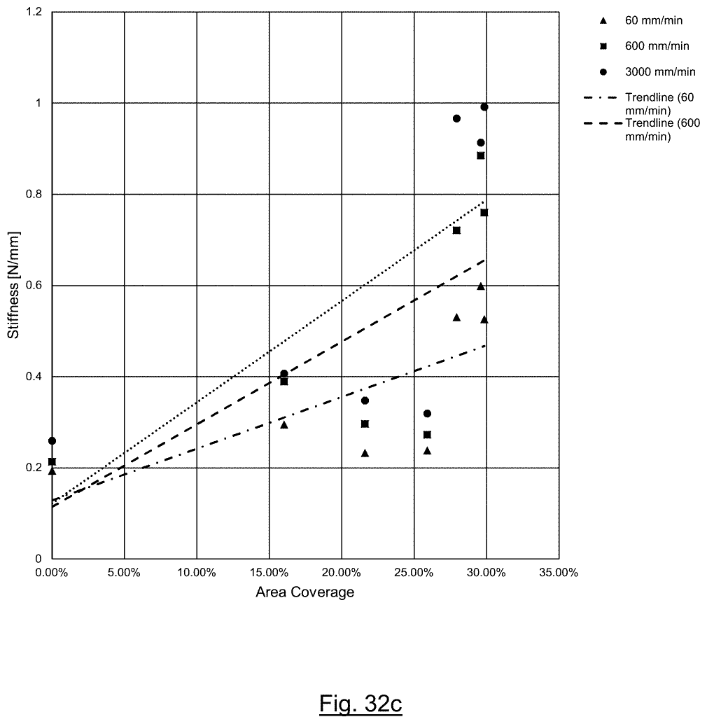

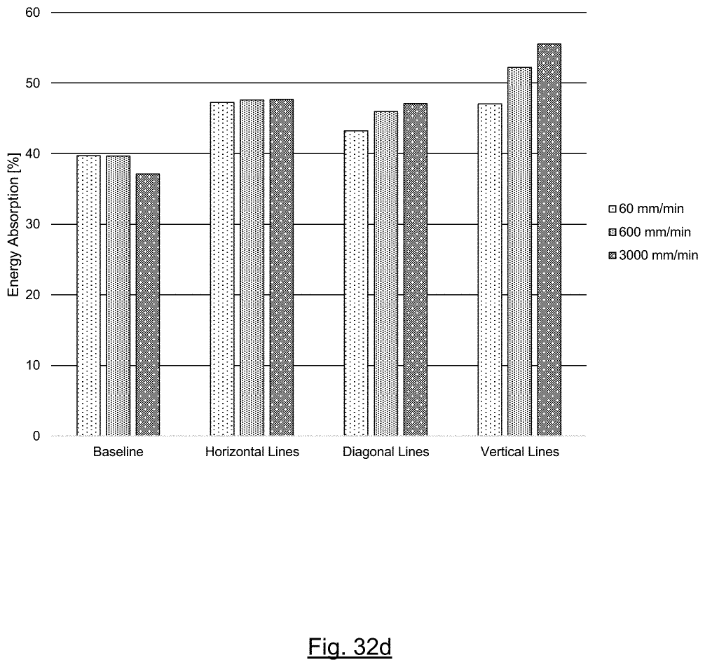

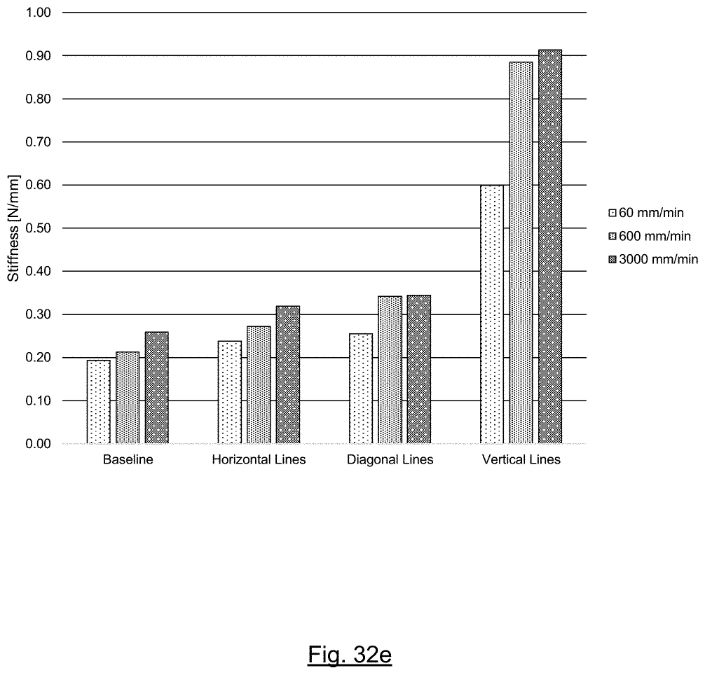

FIG. 32 shows a chart of the axial RMS displacement of the breast tissue of a user when wearing sports bras according to embodiments of the present disclosure whilst star jumping. FIG. 32 shows that, for star jumping (a high impact activity), vertical lines reduced axial displacement by 22%. Incorporating vertical locking features improved this by a further 12% to 34%. The horizontal lines had a negligible effect on axial breast displacement while star jumping.

FIG. 32a shows a chart of the axial RMS displacement of the breast tissue of a user when wearing sports bras according to embodiments of the present disclosure whilst running at 10 kph. FIG. 32a shows that providing a sport bra with motion controlling cells in a Voronoi grid reduces the axial RMS displacement of the breast tissue of the user. Furthermore, FIG. 32a also shows that a greater reduction in displacement is achieved when the strain-rate sensitive material is attached to the inside of the sports bra. The reduction in displacement (compared to the baseline sports bra) when the strain-rate sensitive material is attached to the outside of the sports bra is 26.61%. The reduction in displacement (compared to the baseline sports bra) when the strain-rate sensitive material is attached to the inside of the sports bra is 35.87%. It is believed that the greater reduction in displacement when the strain-rate sensitive material is attached to the inside of the sports bra is due to the SRS material being positioned closer to the user's body.

FIG. 32b shows a graph of the energy absorption of motion control tapes according to embodiments of the present disclosure having differing area coverage of SRS material at a number of stretch speeds. FIG. 32b shows that (a) energy absorption increases with the coverage area of the energy tape and (b) as area coverage increases, the increase in energy absorption which occurs as stretch speed increases becomes more pronounced.

FIG. 32c shows a graph of the stiffness of motion control tapes according to embodiments of the present disclosure having differing area coverage of SRS material at a number of stretch speeds. FIG. 32c shows that (a) stiffness increases with the coverage area of the energy tape and (b) as area coverage increases, the increase in stiffness which occurs as stretch speed increases becomes more pronounced.

FIG. 32d shows a chart of the energy absorption of motion control tape according to embodiments of the present disclosure at a number of stretch speeds. FIG. 32d compares energy absorption by motion control tape having motion controlling cells formed as lines of SRS material in different directions. In this case, the vertical direction is the direction in which the tape is stretched. FIG. 32d shows that energy absorption increases the more aligned the lines are to the direction of stretch.

FIG. 32e shows a chart of the stiffness of motion control tape according to embodiments of the present disclosure at a number of stretch speeds. FIG. 32e compares stiffness by motion control tape having motion controlling cells formed as lines of SRS material in different directions. In this case, the vertical direction is the direction in which the tape is stretched. FIG. 32e shows that stiffness increases the more aligned the lines are to the direction of stretch.

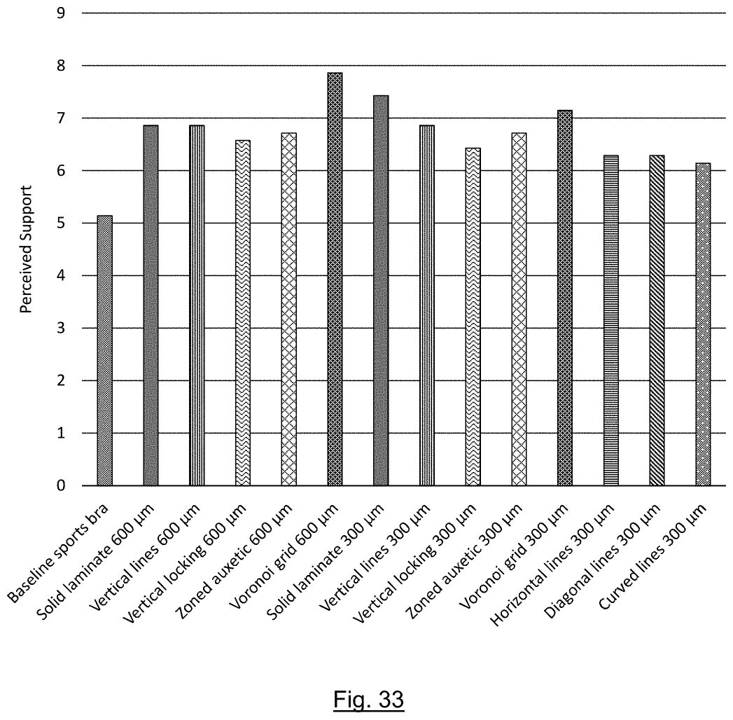

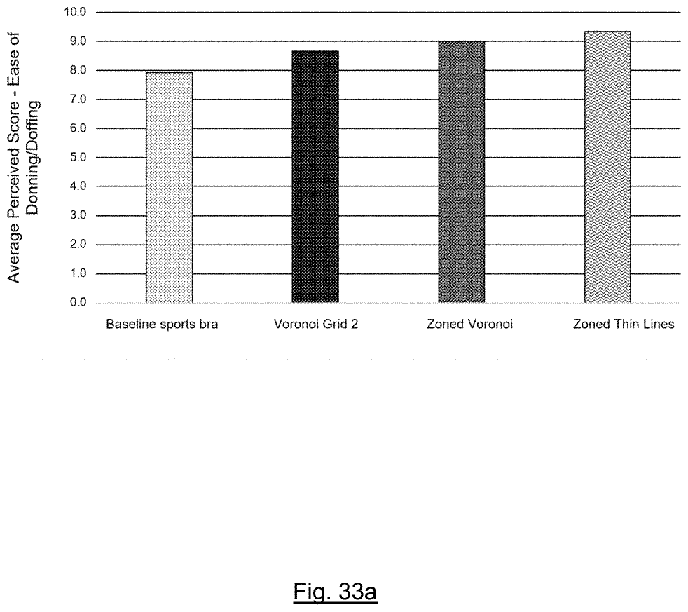

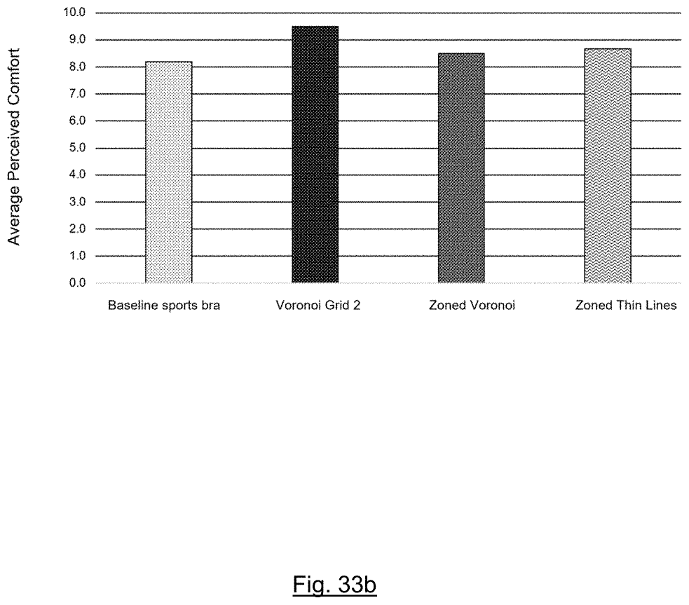

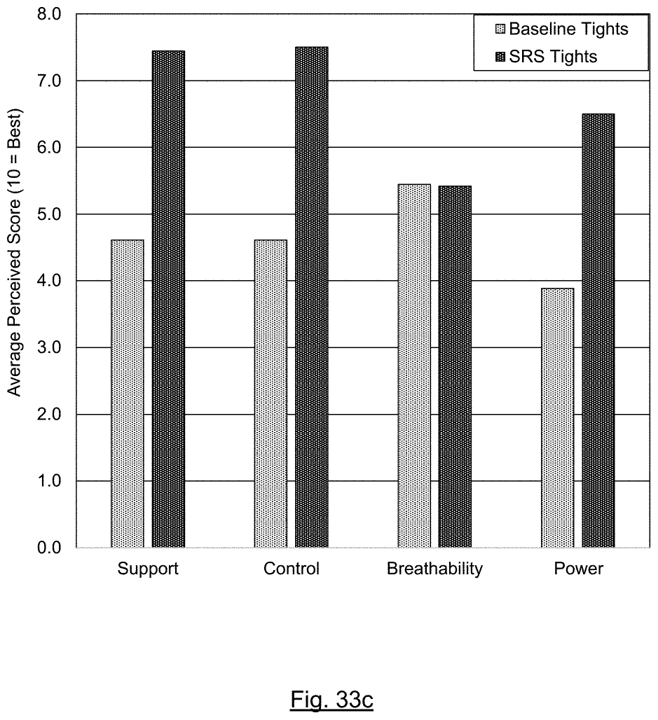

FIG. 33 shows the perceived support given by sports bras according to embodiments of the present disclosure. This graph shows that perceived support increases with addition of strain-rate sensitive substance.

FIG. 33a shows a chart of the average perceived score for ease of donning and doffing of sports bras according to embodiments of the present disclosure. The scores were obtained by asking subjects to conduct physical activity in the sports bra and then rank the ease of donning and doffing in a subjective feedback questionnaire on a scale of 1 (low/poor) to 10 (high/best). FIG. 33a shows that sports bras having motion control systems according to embodiments of the present disclosure were perceived to be easier to don and doff compared to the baseline sports bra.

FIG. 33b shows a chart of the average perceived comfort of sports bras according to embodiments of the present disclosure. The scores were obtained by asking subjects to conduct physical activity in the sports bra and then rank the comfort in a subjective feedback questionnaire on a scale of 1 (low/poor) to 10 (high/best). FIG. 33b shows that sports bras having motion control systems according to embodiments of the present disclosure were perceived to be more comfortable than the baseline sports bra.

FIG. 33c shows a chart of the perceived support, comfort, breathability, and power of tights according to embodiments of the present disclosure. The results were obtained by having subjects conduct a physical activity in the compression tights garments and then rank perceived support, control, breathability, and power in a subjective feedback questionnaire on the scale of 1 (low/poor) to 10 (high/best). FIG. 33c shows that the tights including a motion control system provide improved perceived support, control, and power.

Table 9 shows a comparison of the baseline sports bra with sports bras having motion controlling cells formed as a solid laminate and as a Voronoi grid according to embodiments of the present disclosure. Table 9 shows: (a) axial RMS displacement [mm], (b) improvement in axial displacement [%], (c) circumferential RMS displacement [mm], (d) improvement in circumferential displacement [%], and (e) area coverage of the SRS material [cm²].

TABLE 9

| Sample Name | (a) | (b) | (c) | (d) | (e) |
|---|---|---|---|---|---|
| Baseline sports bra | 8.93 | — | 14.06 | — | |
| Solid laminate | 3.32 | 63% | 7.02 | 50% | 570.87 |
| Voronoi grid | 4.79 | 46% | 9.26 | 34% | 408.58 |

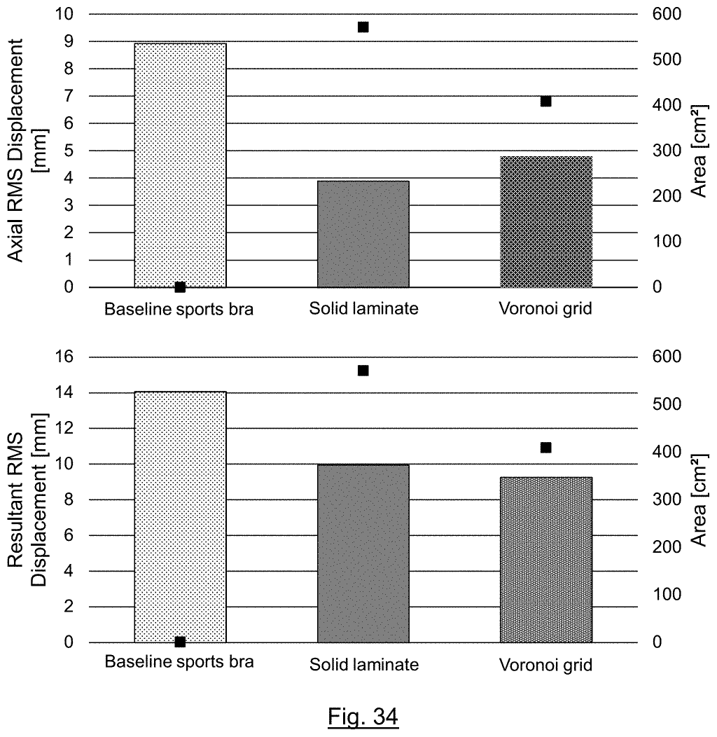

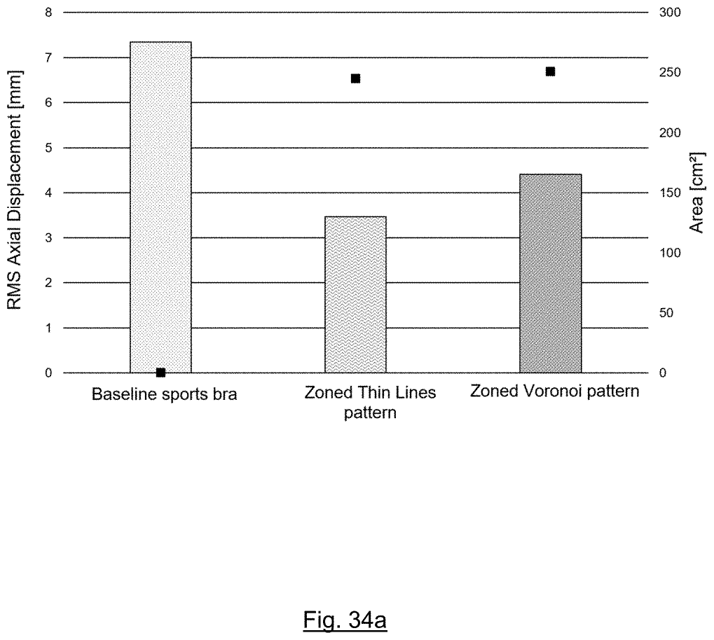

Table 9 shows that SRS material laminated to the sports bra can reduce the vertical and resultant displacement of the breast. The solid laminate provides 56% improvement in vertical RMS displacement compared to the baseline sports bra with the coverage area of 570.87 cm², while the Voronoi grid pattern provides 46% improvement in vertical RMS displacement with a coverage area of 408.58 cm² (28% less area coverage). FIG. 34 shows graphs of the axial and resultant RMS displacements of the breast tissue of a user when wearing sports bras according to embodiments of the present disclosure, along with (black square dots indicating) the total area of strain rate sensitive material in each bra (as listed in Table 9).

FIG. 34a shows a chart of axial RMS displacement of the breast tissue of a user when wearing sports bras according to embodiments of the present disclosure and the area coverage (denoted by black square dots) of strain-rate sensitive material for those sports bras. FIG. 34a shows that both a zoned Voronoi pattern and a zoned thin lines pattern provided a reduction in axial RMS displacement. The zoned Voronoi pattern provided a 35.87% reduction. The zoned thin lines pattern provided a 52.74% reduction. Both the zoned Voronoi pattern and the zoned thin lines pattern provide a similar area coverage of SRS material of approximately 250 cm².

Table 10 shows the (a) RMS axial displacement in mm and (b) improvement in RMS axial displacement (as a percentage) of the breast of a user wearing sports bras according to embodiments of the present disclosure. Table 10 compares a sports bra having a single 300 μm thick layer of SRS material with a sports bra having two adjacent 150 μm thick layers of SRS material arranged such that the two layers can move and rub against one another. The amount of SRS substance in each garment is the same, but Table 10 shows, whilst both the single layer constructions and the double layer construction provide a reduction in RMS axial displacement compared to the baseline sports bra, the 2-layer construction provides a greater reduction in RMS axial displacement than the single layer construction.

TABLE 10

| Sample Name | (a) | (b) |
|---|---|---|
| Baseline sports bra | 7.34 | 0.00% |
| Zoned Voronoi pattern 300 μm - 1 layer | 4.41 | 39.86% |
| Zoned Voronoi pattern 150 μm - 2 layer | 3.64 | 50.44% |

In embodiments, the SRS is laminated onto the garment as a density “mapped” pattern. Table 11 shows the (a) RMS axial displacement in mm and (b) improvement in RMS axial displacement (as a percentage) of the breast of a user wearing sports bras according to such embodiments. Table 11 compares sports bras having motion controlling cells in a zoned Voronoi pattern, whereby one of the sports bras has SRS in a zoned Voronoi “extended” pattern which provides additional zonal support across the lower neck and rib cage area.

TABLE 11

| Sample Name | (a) | (b) |
|---|---|---|
| Baseline sports bra | 7.34 | 0.00% |
| Zoned Voronoi pattern | 4.41 | 39.86% |
| Zoned Voronoi Extended pattern | 4.02 | 45.24% |

Table 11 shows that the additional zonal supports of the zoned Voronoi “extended” pattern provide better control of the soft tissue, with RMS axial displacement of the breast being reduced by 45.24% compared to the 39.86% reduction provided by the standard zoned Voronoi pattern without additional zonal support. Thus, zonal mapping of the SRS patterns can improve the motion control system performance by strategically affecting the garment stiffness and damping properties.

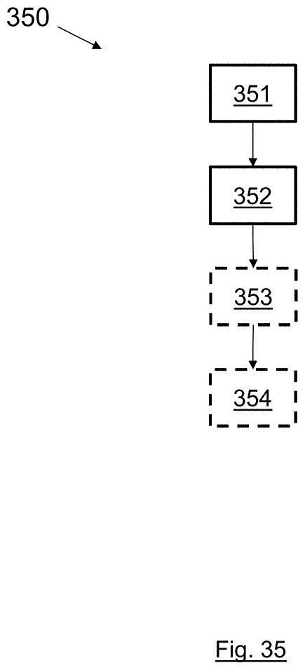

FIG. 35 shows a flow chart illustrating the steps of a method 350 of manufacturing a wearable item comprising a motion control system according to embodiments of the present disclosure. A first step of the method, represented by item 351, comprises forming the wearable item comprising a body-close wearable item which, when worn by a user, at least a part of the wearable item is positioned adjacent to the body of the user. A second step of the method, represented by item 352, comprises forming the motion control system comprising at least one layer of strain-rate sensitive material configured to control motion of one or more body parts of the user.

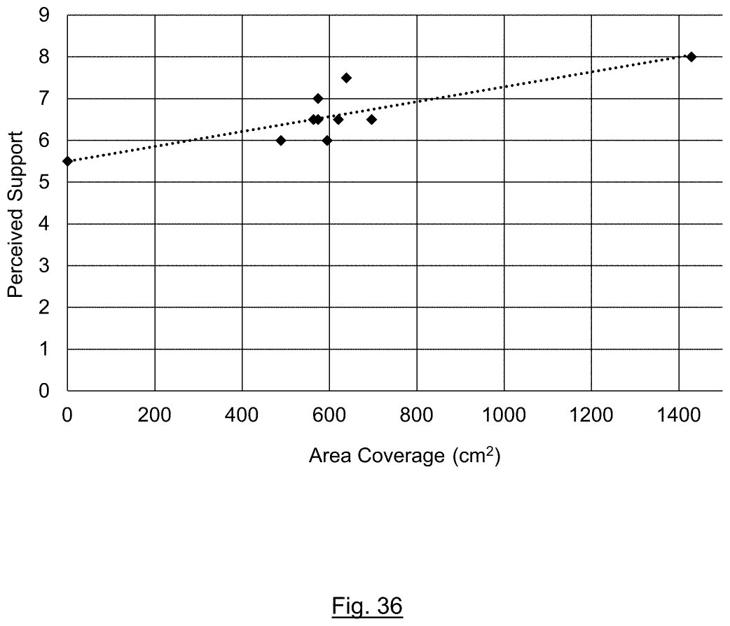

FIG. 36 shows a graph of perceived support of garments compared to the area coverage of material according to embodiments. It is demonstrated that as area coverage of material increases, perceived support also increases (implied by the positive gradient of the best-fit line).

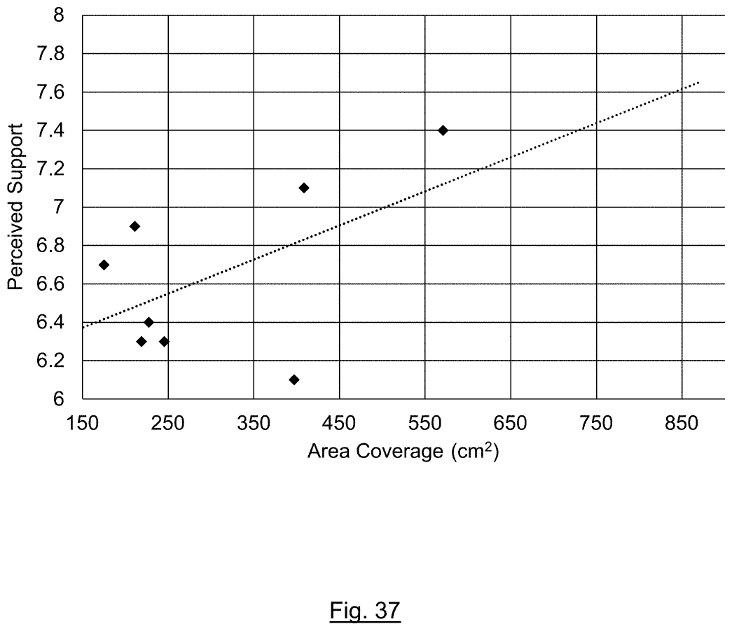

FIG. 37 shows a graph of perceived support of the sports bras compared to the area coverage of material. It is demonstrated that as area coverage of material increases, perceived support also increases (implied by the positive gradient of the best-fit line).

In embodiments, the wearable item comprises a textile layer and forming the motion control system comprises attaching a layer of strain-rate sensitive material to the textile layer. In embodiments, the attaching comprises laminating the layer of strain-rate sensitive material to the textile layer. In embodiments, the attaching comprises adhering the layer of strain-rate sensitive material to the textile layer. In embodiments, the attaching comprises weaving the layer of strain-rate sensitive material into the textile layer. In embodiments, the attaching comprises heat-pressing the layer of strain-rate sensitive material onto the textile layer.

In embodiments, the SRS material is formed by extrusion into a film (for example, up to 1 mm thick). This film is then cut into one or more panels having the desired geometry. In embodiments, these panels are then heat pressed, laminated, adhered, sewn, knitted, welded, impregnated, or coated onto the textile layer.

In embodiments, the SRS material is extruded into a foam in the desired geometry on panels. In embodiments, those panels are then heat pressed, laminated, or adhered to the textile layer.

In embodiments, the SRS material is extruded into filaments/fibres. In such embodiments, these SRS filaments are blended with a synthetic fibre to make an active stretch yarn blend. In embodiments, the yarn is knitted into the garment. In such embodiments, the yarn may be composed of 10-20% SRS material and 80-90% synthetic fibre. Alternatively additionally, the yarn may be woven into the garment. In such embodiments, the yarn may be composed of 40-50% SRS material and 50-60% synthetic fibre. The knits/weave can be made such that there is a greater density of SRS material in certain places (i.e. different geometries) in order to provide targeted compression.

An optional third step of the method, represented by item 353, comprises attaching a further layer of strain-rate sensitive material to the textile layer. In embodiments, the further layer is attached to an opposing side of the textile layer to the first layer, such that the textile layer is sandwiched in-between the two layers of strain-rate sensitive material.

An optional fourth step of the method, represented by item 354, comprises attaching a further textile layer to the layer of strain-rate sensitive material. In embodiments, the further textile layer is attached to an opposing side of the layer of strain-rate sensitive material to the first textile layer, such that strain-rate sensitive material layer is sandwiched in-between the two textile layers.

Whilst the present disclosure has been described and illustrated with reference to particular embodiments, it will be appreciated by those of ordinary skill in the art that the disclosure lends itself to many different variations not specifically illustrated herein. By way of example only, certain possible variations will now be described.

Although the present disclosure has been described embodied as a sports bra, shorts, and kinesiology tape, it will be appreciated by the skilled person that other wearable items are also possible. For example, the wearable item may comprise a sock, a sleeve (i.e. an open-ended tube), leggings, gloves, or stockings. The wearable item may, for example, comprise a shoe, with the motion control system acting as a substitute for shoelaces.

Similarly, although the benefits of embodiments of the present disclosure have been described primarily in a sporting context, it will be appreciated that garments providing active control of the motion of body parts also find use in other settings (for example, as medical compression garments for use in physical therapy or as shapewear).

Although a number of geometries of motion controlling cells have been described, it will be appreciated that other geometries not explicitly described are also possible and, in some cases, desirable. It will be understood by the skilled person that the specific geometries of the motion controlling cells are tailored to the desired constraints to be placed on movement of the user's body parts (i.e. extent to which motion in any given direction is to be controlled or not).

The present disclosure also provides a wearable item comprising a layer of active material, wherein the wearable item comprises a body-close wearable item which, when worn by a user, at least a part of the wearable item is positioned adjacent to the body of the user, and wherein the layer of active material comprises at least one layer of strain-rate sensitive material configured to control motion of one or more body parts of the user.

It will be appreciated that an active material is a material which has dynamic (for example, changing in response to strain rate) stiffness and/or damping properties.

Where in the foregoing description, integers or elements are mentioned which have known, obvious or foreseeable equivalents, then such equivalents are herein incorporated as if individually set forth. Reference should be made to the claims for determining the true scope of the present disclosure, which should be construed so as to encompass any such equivalents. It will also be appreciated by the reader that integers or features of the disclosure that are described as preferable, advantageous, convenient or the like are optional and do not limit the scope of the independent claims. Moreover, it is to be understood that such optional integers or features, whilst of possible benefit in some embodiments of the disclosure, may not be desirable, and may therefore be absent, in other embodiments.

What is claimed is:

1. A wearable item comprising a motion control system,
wherein the wearable item comprises a body-close wearable item which, when worn by a user, at least a part of the wearable item is positioned adjacent to a body of the user, and
wherein the motion control system comprises at least one layer of strain-rate sensitive material configured to control motion of one or more body parts of the user.

2. The wearable item according to claim 1, wherein the at least one layer of strain-rate sensitive material comprises a solid strain-rate sensitive material, and/or
wherein the at least one layer of strain-rate sensitive material comprises a chemically strain-rate sensitive material, and/or
wherein the at least one layer of strain-rate sensitive material comprises a polymer, and/or
wherein the at least one layer of strain-rate sensitive material comprises a chemical dilatant.

3. The wearable item according to claim 1, wherein:
the one or more body parts comprise soft-tissue body parts, and
the controlling comprises controlling velocity, displacement and/or acceleration of the soft-tissue body parts.

4. The wearable item according to claim 1, wherein the controlling comprises controlling energy absorption.

5. The wearable item according to claim 1, wherein the controlling comprises controlling stiffness.

6. The wearable item according to claim 3, wherein the controlling is dependent on a frequency of motion of the soft-tissue body parts.

7. The wearable item according to claim 6, wherein the controlling is greater at relatively high frequencies of motion of the soft-tissue body parts compared to relatively low frequencies of motion of the soft-tissue body parts.

8. The wearable item according to claim 7, wherein the controlling comprises performing substantially zero control at relatively low frequencies of motion of the soft-tissue body parts.

9. The wearable item according to claim 7, wherein:
the relatively low frequencies comprise frequencies below 5 Hz and the relatively high frequencies comprise frequencies above 5 Hz, or
the relatively low frequencies comprise frequencies between 1 Hz and 5 Hz and the relatively high frequencies comprise frequencies between 10 Hz and 30 Hz.

10. The wearable item according to claim 1, wherein the at least one layer of strain-rate sensitive material is configured to control motion of the one or more body parts of the user in a given direction.

11. The wearable item according to claim 10, wherein the given direction comprises one or more of:
a radial direction from a bone of the user, and
a circumferential direction around a bone of the user.

12. The wearable item according to claim 10, wherein the given direction comprises one or more of:
a radial direction from a given body part of the user,
an axial direction along a given body part of the user, and
a circumferential direction around a given body part of the user.

13. The wearable item according to claim 1, wherein the at least one layer of strain-rate sensitive material is configured not to control motion of the one or more body parts of the user in a different, given direction.

14. The wearable item according to claim 13, wherein the different, given direction comprises an axial direction along a bone of the user.

15. The wearable item according to claim 1, comprising:
a textile layer,
wherein the at least one layer of strain-rate sensitive material is attached to the textile layer.

16. The wearable item according to claim 15, wherein the at least one layer of strain-rate sensitive material is laminated to the textile layer, and/or
wherein the at least one layer of strain-rate sensitive material is adhered to the textile layer, and/or
wherein the at least one layer of strain-rate sensitive material is woven and/or knitted into the textile layer, and/or
wherein the at least one layer of strain-rate sensitive material is heat-pressed onto the textile layer.

17. The wearable item according to claim 1, wherein the at least one layer of strain-rate sensitive material comprises a continuous sheet.

18. The wearable item according to claim 1, wherein the at least one layer of strain-rate sensitive material comprises strain-rate sensitive filament material.

19. The wearable item according to claim 1, wherein the at least one layer of strain-rate sensitive material comprises a yarn blend of strain-rate sensitive fiber material and synthetic fiber material.

20. The wearable item according to claim 1, wherein the at least one layer of strain-rate sensitive material comprises a plurality of planar motion controlling cells.

21. The wearable item according to claim 20, wherein at least one of the plurality of planar motion controlling cells comprises one or more of the following geometries:
diagonal lines,
vertical lines,
horizontal lines,
curved lines,
squares,
diamonds,
triangles,
hexagons, and
auxetic polygons.

22. The wearable item according to claim 20, wherein at least one of the plurality of planar motion controlling cells comprises a geometry determined by a surface tessellation process.

23. The wearable item according to claim 22, wherein the surface tessellation process comprises a Voronoi tessellation process.

24. The wearable item according to claim 20, wherein:
the plurality of planar motion controlling cells comprises a first subset of motion controlling cells having a first geometry and a second subset of motion controlling cells, different from the first subset of motion controlling cells, having a second geometry, different from the first geometry, and
the motion controlling cells in the first subset have different motion control properties from motion controlling cells in the second subset.

25. The wearable item according to claim 24, wherein:
motion controlling cells in the first subset are located in a first zone of the wearable item, and
motion controlling cells in the second subset are located in a second, different zone of the wearable item.

26. The wearable item according to claim 11, wherein the wearable item comprises a pair of shorts and the bone of the user comprises a femur.

27. The wearable item according to claim 12, wherein the wearable item comprises a brassiere and the given body part of the user comprises a torso of the user.

28. The wearable item according to claim 1, wherein the wearable item comprises a sock.

29. The wearable item according to claim 1, wherein the wearable item comprises a sleeve or tube with an opening at both ends.

30. The wearable item according to claim 1, wherein:
the motion control system comprises first and second layers of strain-rate sensitive material configured to control motion of one or more body parts of the user, and
wherein the first and second layers of strain-rate sensitive material are positioned adjacent to each other.

31. The wearable item according to claim 15, wherein:
the motion control system comprises first and second layers of strain-rate sensitive material configured to control motion of one or more body parts of the user, and
the textile layer is sandwiched in-between the first and second layers of strain-rate sensitive material.

32. The wearable item according to claim 15, wherein:
the motion control system comprises a further textile layer, and
the at least one layer of strain-rate sensitive material layer is sandwiched in-between the textile layer and the further textile layer.

33. A method of manufacturing a wearable item comprising a motion control system, the method comprising:
forming the wearable item comprising a body-close wearable item which, when worn by a user, at least a part of the wearable item is positioned adjacent to a body of the user; and
forming the motion control system comprising at least one layer of strain-rate sensitive material configured to control motion of one or more body parts of the user.
